(19) United States
(12) Patent Application Publication
(10) Pub. No.: US 2022/0218759 A1
(43) Pub. Date: Jul. 14, 2022

(54) COMPOSITIONS AND METHODS FOR IMPROVING TREATMENT OUTCOMES FOR PATIENTS HAVING HEMATOLOGICAL MALIGNANCIES USING AN EXPANDED STEM CELL PRODUCT

(71) Applicant: Deverra Therapeutics Inc., Seattle, WA (US)

(72) Inventor: Colleen Delaney, Seattle, WA (US)

(73) Assignee: Deverra Therapeutics Inc., Seattle, WA (US)

(21) Appl. No.: 17/611,829

(22) PCT Filed: May 15, 2020
(86) PCT No.: PCT/US2020/033182

§ 371 Date: Nov. 16, 2021

Related U.S. Application Data
(60) Provisional application No. 62/852,147, filed on May 23, 2019.
(60) Provisional application No. 62/849,588, filed on May 17, 2019.

(51) Int. Cl. A61K 35/51; A61P 35/02; A61K 35/28; A61K 31/7068; A61K 31/65; A61K 31/704; A61K 31/7076; A61K 38/19; A61K 31/7048; A61K 31/136; C12N 5/0775

(52) U.S. Cl. CPC A61K 35/51; C12N 2501/2306; A61K 35/28; A61K 31/7068; A61K 31/65; A61K 31/704; A61K 31/7076; A61K 38/193; A61K 31/7048; A61K 31/136; C12N 5/0665; C12N 2501/125; C12N 2501/145; C12N 2501/2303; A61P 35/02

(57) ABSTRACT

The present invention relates to methods and compositions for treating a hematological malignancy with an expanded hematopoietic stem cell product in combination with a chemotherapy regimen.

### CROSS-REFERENCES TO RELATED APPLICATIONS

This application claims the benefit of U.S. Provisional Application No. 62/849,588, filed May 17, 2019, and U.S. Provisional Application No. 62/852,147, filed May 23, 2019, the disclosures of which are incorporated herein by reference in their entirety.

### FIELD OF THE INVENTION

The present invention relates to methods and compositions for improving the treatment outcome of a patient having acute myeloid leukemia (AML) or another hematological malignancy. The expanded stem cell product comprises hematopoietic stem cells or hematopoietic stem and progenitor cells derived from multiple donors that are combined (e.g., pooled) without matching (i.e., without regard to) the HLA-type of the cord blood units to each other or to the HLA-type of the patient. The expanded stem cell product can be administered following a chemotherapy regimen, such as an induction regimen, salvage regimen or consolidation regimen of varying intensity.

### BACKGROUND

Acute myeloid leukemia (AML) is a leading cause of adult acute leukemia and accounts for a majority of all adult leukemias. Despite extensive research, AML is associated with low long-term survival; the 5-year overall survival rates are about 28.3% of all patients (SEER) and about 24% for patients 20 years and older. In contrast, for patients younger than 20 years of age, the 5-year overall survival rate is about 67%. Conventional chemotherapy can effectively achieve initial remission of the disease in some AML patients. However, due to the highly heterogeneous nature of the disease, about 30% of AML patients do not respond to chemotherapy. It is important to note that chemotherapy fails to achieve complete clearance of the disease in most patients, and more than 70% of patients in remission suffer from relapsing AML within 2 years after the initial treatment. There is currently no standard treatment regimen for patients with relapsed AML, which is associated with poor prognosis. Relapsed AML can be caused by a phenomenon called minimal residual disease (MRD), which is mediated by an AML cell population with resistance to chemotherapy. MRD is proposed to be mediated by a leukemic stem cell (LSC) population, as this cell population has the ability to withstand chemotherapy and other treatments. Therefore, development of treatments to target AML and address MRD to achieve relapse-free clearance of the disease has been an active area of research.

Allogeneic hematopoietic stem cell transplantation (allo-HSCT) has been investigated as a curative treatment option for AML patients and has been associated with higher disease-free survival rates than conventional chemotherapy. These grafts are commonly derived from bone marrow, peripheral blood, and/or umbilical cord blood, and in particular, with respect to peripheral blood grafts subsequent to stem cell mobilization in the donor by administering, for example, GM-CSF. The cells of the graft are a heterologous mix of blood and immune cells, including stem cells, red cells, white blood cells including T cells, NK cells, and the like, and platelets. Hematopoietic stem cells comprise a very small number of the cells of a hematopoietic stem cell graft, generally less than 1% of the total cell population. Donor-derived T cell mediated anti-leukemic effects contribute to the increased survival in patients, as autologous and T cell depleted grafts have been reported to be associated with higher relapse rates. The use of allo-HSCT in the clinic has been limited by a shortage of suitably HLA-matched donors and has been associated with toxicity and other associated complications Immune responses have been reported to cause normal tissue damage (e.g., graft-versus-host disease (GVHD)).

Micro-transplants (the infusion of a non-engrafting stem cell graft) and/or non-engrafting donor lymphocyte infusions have been investigated as a potential curative treatment for AML patients. Unrelated donor mismatched microtransplantation of mobilized PBSCs from a single donor was reported to provide some benefit to a patient, but the patient went on to relapse. (Punwani et al., 2018, Leuk. Res. Rep. 9:18-20.) HLA-mismatched allogenic cellular therapy has also been investigated for treatment of AML using partially matched mobilized peripheral blood cells. (Mohrbacher et al., 2014, Blood 124:5944.) Five of eight patients were reported to achieve a complete remission/complete remission with incomplete hematologic recovery (CR/Cri) lasting 3 to 10+ months, although the authors reported the responses were not as durable as hoped for, despite partial matching of the transplants.

Therefore, there remains a need for the development of treatments to target AML, and other hematological malignancies to achieve relapse-free clearance of the disease. There also remains a need in the art to develop less toxic therapies and to improve treatment outcomes using existing treatment regimens for patients having AML, including relapsed/refractory AML, de novo AML, and treatment-related AML, as well as other hematological malignancies, such as myelodysplastic syndrome (MDS), a myeloproliferative neoplasm (MPN) and non-Hodgkin Lymphoma (NHL).

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present invention provides methods for improving treatment outcome for a patient having acute myeloid leukemia (AML) or other hematological malignancy by administering a chemotherapy regimen, or a cycle thereof to a patient in need of treatment, and then administering a fixed dose of an expanded stem cell product to the patient, wherein the administering is done without matching the HLA-type of the expanded stem cell product to the HLA type of the patient. The expanded stem cell product is a cell-based product derived from the pooling of hematopoietic stem cells or hematopoietic stem and progenitor cells from at least two human donors, wherein the hematopoietic stem cells or hematopoietic stem and progenitor cells from the donors are combined without matching to the HLA type of the other donors and without matching to the HLA type of the patient. The expanded stem cell product is depleted of T cells and red blood cells.

Also provided are methods for improving treatment outcome for a human patient having AML or other hematological malignancy, comprising: (a) selecting an expanded hematopoietic stem cell product for administration to the patient, wherein the selecting does not take into account (i.e., without matching) the HLA-type of the expanded stem cell product or the HLA-type of the patient; (b) administering a chemotherapy regimen, or a cycle thereof, to the patient; and (c) administering a fixed dose of the selected expanded stem cell product to the patient. The expanded stem cell product is a cell based product derived from the pooling of hematopoietic stem cells or hematopoietic stem and progenitor cells from at least two human donors, wherein the hematopoietic stem cells or hematopoietic stem and progenitor cells from the donors are pooled without matching to the HLA type of the other donors and without matching to the HLA type of the patient. As above, the expanded stem cell product is depleted of T cells and red blood cells.

The present invention further provides methods for treating a patient having AML or other hematological malignancy by administering a chemotherapy regimen, or a cycle thereof, to the patient, and then administering a fixed dose of an expanded stem cell product to the patient, wherein the administering is done without matching the HLA-type of the expanded stem cell product to the HLA type of the patient. The expanded stem cell product is a cell-based product derived from the pooling of hematopoietic stem cells or hematopoietic stem and progenitor cells from at least two human donors, wherein the hematopoietic stem cells or hematopoietic stem and progenitors cells from the donors are pooled without matching to the HLA type of the other donors and without matching to the HLA type of the patient. As above, the expanded stem cell product is depleted of T cells and red blood cells.

In certain embodiments, a fixed dose of the expanded stem cell product contains from about 50 to about 400 million viable CD34+ cells. In certain embodiments, a fixed dose of the expanded stem cell product contains about 50 million, about 75 million, about 100 million, about 200 million, about 300 million, or about 400 million viable CD34+ cells.

In some embodiments, the expanded stem cell product is prepared, cryopreserved and stored for later use as an “off the shelf” product. The cryopreserved expanded stem cell product is thawed prior to administering to the patient.

The expanded stem cell product is a pool of at least two expanded hematopoietic stem cell populations and/or at least two expanded hematopoietic stem and progenitor cell populations, wherein each cell population is derived from a separate donor. In some embodiments, each cell population is obtained from a separate cord blood unit or placental blood unit (i.e., from a different human at birth). The HLA-types of the at least two cell populations in the pool are not HLA-matched to each other. Optionally, the expanded stem cell product is a pool of two or more hematopoietic stem cell populations or hematopoietic stem and progenitor cell populations that are pooled prior to expansion, which pool is then expanded, or the cell populations are pooled after expansion. Optionally, the expanded stem cell product is a pool of two or more human cord blood or placental blood stem cell populations or stem and progenitor cell populations that are pooled prior to expansion, which pool is then expanded, or the cell populations are pooled after expansion. In one embodiment, the cell populations in the pool are all derived from umbilical cord blood and/or placental blood of individuals of the same race, e.g., African-American, Caucasian, Asian, Hispanic, Native-American, Australian Aboriginal, Inuit, Pacific Islander, or are all derived from umbilical cord blood and/or placental blood of individuals of the same ethnicity, e.g., Irish, Italian, Indian, Japanese, Chinese, Russian, and the like. In another embodiment, the hematopoietic stem cells or hematopoietic stem and progenitor cells in the pool are combined without regard to either race or ethnicity.

In yet another embodiment, the method of improving treatment outcome for a patent having AML or another hematological malignancy comprises, prior to said administering, a step of expanding ex vivo isolated human cord blood stem cells, or stem and progenitor cells, obtained from the umbilical cord blood and/or placental blood of at least two humans at birth. Preferably, the expanding step comprises contacting the human cord blood stem cells, or stem and progenitor cells, with an agonist of Notch function. The agonist can be a Delta protein or a Serrate protein, or a fragment of a Delta protein or Serrate protein, which fragment is able to bind a Notch protein. In another embodiment, the expanding step comprises contacting the hematopoietic stem cells, or stem and progenitor cells, with Delta1^{ext-IgG} (DXI).

In another embodiment, a method for improving treatment outcome for a human patient having a hematological malignancy is provided, which method comprises: (a) enriching for hematopoietic stem cells, or hematopoietic stem and progenitor cells, from isolated human cord blood stem cells or stem and progenitor cells obtained from the umbilical cord blood and/or placental blood at least two humans at birth to produce a cell population enriched for hematopoietic stem cells or hematopoietic stem and progenitor cells; (b) expanding ex vivo the cell population enriched for hematopoietic stem cells or hematopoietic stem and progenitor cells to produce an expanded stem cell product; (c) administering a chemotherapy regimen or a cycle thereof to the patient; and (d) administering a fixed dose of the expanded stem cell product to a human patient in need thereof, wherein the administering is done without matching the HLA type of the expanded hematopoietic stem cells or expanded hematopoietic stem and progenitor cells of the expanded stem cell product to the HLA-type of the patient and without matching the HLA type of the expanded hematopoietic stem cells or expanded hematopoietic stem and progenitor cells of the expanded stem cell product to each other. In a preferred embodiment, the expanded hematopoietic stem cells are CD34+ cells. This method can further comprise the steps of freezing and storing the expanded stem cell product after step (b) and thawing the expanded stem cell product before step (c). In certain embodiments, the patient suffers from AML, such as de novo AML, relapsed/refractory AML or treatment related AML, or other hematological malignancy such as Non-Hodgkin lymphoma, myelodysplastic syndrome (MDS) or a myeloproliferative neoplasm (MPN).

### Definitions

Although any methods and materials similar or equivalent to those described herein can be used in the practice or testing of the present invention, the preferred methods and materials are described. For purposes of the present invention, the following terms are defined below.

As used herein, an “expanded stem cell product” refers to cell populations enriched for hematopoietic stem cells or stem and progenitor cells that have been subjected to a technique for expanding the hematopoietic stem cells, or hematopoietic stem and progenitor cells of the cell populations, which technique has been shown to result in (i) an increase in the number of hematopoietic stem cells, or hematopoietic stem and progenitor cells, in an aliquot of the cells thus expanded, or (ii) an increased number of severe-combined-immunodeficiency (SCID) repopulating cells determined by limiting-dilution analysis as shown by enhanced engraftment in non-obese diabetic/severe-combined-immunodeficiency (NOD/SCID) mice infused with an aliquot of the cells thus expanded; relative to that seen with an aliquot of the cells that is not subjected to the expansion technique. (See U.S. Patent Publication No. 2013/0095079; Delaney et al., 2010, Nature Med. 16(2):232-236.) Typically, the hematopoietic stem cells or hematopoietic stem and progenitor cells are CD34+. In some embodiments, the hematopoietic stem cells or hematopoietic stem and progenitor cells are derived from human umbilical cord blood and/or human placental blood. In some embodiments, the expanded stem cell product is prepared using a Notch-agonist expansion method. In some embodiments, the expanded stem cell product is prepared using a DXI expansion method. The expanded stem cell product is depleted of T cells and red blood cells.

As used herein, a “chemotherapy regimen” refers to a regimen for chemotherapy, defining the drugs to be used, their dosage, the frequency and duration of treatments, and other considerations. Such regimens may combine several chemotherapy drugs in combination chemotherapy. The majority of drugs used in chemotherapy are cytostatic or cytotoxic.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates a flow chart demonstrating an exemplary procedure for enriching a population of CD34+ cells and expanding the enriched cell population.

FIG. 2 shows the subject disposition during a clinical study described in Example 3.

### DETAILED DESCRIPTION

While illustrative embodiments have been illustrated and described, it will be appreciated that various changes can be made therein without departing from the spirit and scope of the invention.

Hematopoietic stem/progenitor cell transplantation, particularly autologous hematopoietic stem/progenitor cell transplant is generally performed to rescue bone marrow aplasia following high-dose chemotherapy for solid tumors or multiple myeloma. Allogeneic hematopoietic stem cell transplant has been found to be useful in curing leukemias and other hematopoietic malignancies by eradicating the diseased blood and immune system and restoring hematological homeostasis by infusion of a healthy donor hematopoietic stem cell graft. One of the persistent issues in allogeneic hematopoietic stem cell transplantation has been the lack of available allogeneic donors having sufficient HLA antigen and/or allele matching with a patient for successful treatment. More recently, methods and compositions have been devised for providing hematopoietic function in immunocompromised human patients by selecting an expanded hematopoietic stem/progenitor cell sample without taking into account the HLA-type of the expanded human cord blood stem/progenitor cell sample or the HLA-type of the patient. The hematopoietic stem/progenitor cell sample can be used in human patients who are at high risk of morbidity and mortality after undergoing hematopoietic stem cell transplant or high dose chemotherapy to transiently replace or replenish hematopoietic function or to reduce the rate of life-threatening infection. Unexpectedly, an expanded hematopoietic stem cell or hematopoietic stem and progenitor cell product where HLA typing was not carried out and wherein the cell product did not comprise T cells has been found to be useful in treating and/or in increasing the chance of an improved outcome human patients with acute myelogenous leukemia (AML) or certain other hematological malignancies.

The present invention provides methods for treating, and methods for improving a treatment outcome for, a patient having AML or other hematological malignancy by administering a chemotherapy regimen to the patient, followed by administering a fixed dose of an expanded stem cell product to the patient in need thereof, wherein the administering is done without matching the HLA-type of the expanded stem cell product to the HLA-type of the patient. The expanded stem cell product is a cell-based product comprising hematopoietic stem cells or hematopoietic stem and progenitor cells are not HLA matched to each other or to the HLA-type of the patient. In some embodiments, the expanded stem cell product is a pooled product derived from the pooling of hematopoietic stem cells or hematopoietic stem and progenitor cells from cord blood units or placental blood units from at least two different human donors, comprising hematopoietic stems cells or hematopoietic stem and progenitor cells are not HLA-type matched to each other or to the HLA-type of the patient. The phrase “without matching the HLA-type”, “unmatched” or the like, means no steps are taken to have any of the HLA antigens or alleles (HLA-type) match between the patient and the hematopoietic stem cells or hematopoietic stem and progenitor cells in the expanded stem cell product. The selection of the expanded stem cell product is done without matching the HLA-type of the patient to whom the expanded stem cell product will be administered. Similarly, with respect to the source of the hematopoietic stem cells or hematopoietic stem and progenitor cells, e.g., from cord blood units or placental blood units, from which the expanded stem cell product is derived, the phrase “without matching the HLA-type” means no steps are taken to have any of the HLA antigens or alleles (HLA-type) match between the hematopoietic stem cells or hematopoietic stem and progenitor cells in the expanded stem cell product. It should also be noted that the expanded stem cell product is depleted of T cells and red blood cells.

The expanded stem cell product is typically administered after a chemotherapy regimen. The chemotherapy regimen can be a single agent or multi-agent regimen. In some embodiments, the chemotherapy regimen is an induction regimen or a consolidation regimen. An induction regimen comprises the use of chemotherapy as a primary treatment for a patient presenting with advanced cancer for which no alternative treatment exits. A consolidation regimen comprises repetitive cycles of treatment during the immediate post remission period used especially in leukemia. In some embodiments, the chemotherapy regimen is a salvage regimen. A salvage regimen comprises the use of chemotherapy in a patient with recurrence of a malignancy following initial treatment in hope of a cure or prolongation of life. In some embodiments, the expanded stem cell product is administered about 12 to about 48 hours after the chemotherapy regimen, or preferably about 24 to 36 hours after the chemotherapy regimen. In some embodiments, the expanded stem cell product is administered about 12 to about 48 hours after each cycle of the chemotherapy regimen, or preferably about 24 to 36 hours after each cycle, where the chemotherapy regimen is administered in more than one cycle.

In some embodiments, the expanded stem cell product is administered to the patient after the components of a chemotherapy regimen and active metabolites thereof have been cleared from the patient's blood. In some embodiments, the expanded stem cell product is administered to the patient after the components of an induction regimen and active metabolites thereof have been cleared from the patient's blood. In some embodiments, the expanded stem cell product is administered to the patient after the components of a consolidation regimen and active metabolites thereof have been cleared from the patient's blood. In some embodiments, the expanded stem cell product is administered to the patient after the components of a salvage regimen and active metabolites thereof have been cleared from the patient's blood. In some embodiments, where the chemotherapy regimen (such as an induction, salvage, or consolidation regimen) is administered in more than one cycle, the expanded stem cell product is administered to the patient following a cycle or each cycle after the components of the regimen and active metabolites thereof have been cleared from the patient's blood. As used herein, “after . . . regimen and active metabolites thereof have been cleared from the patient's blood” refers to clearance of the components of the regimen (e.g., an induction regimen, a salvage regimen or a consolidation regimen) and active metabolites of those components that would affect the viability of CD34+ stem cells in the patient's blood, such as by decreasing CD34+ stem cell or progenitor cell viability by at least 5%, at least 10%, or at least 20%.

Administration of the expanded stem cell product after each regimen or cycle thereof can improve the treatment outcome of a patient having AML or other hematological malignancy, such as by improving the chances of the patient achieving a remission (e.g., a Complete Remission (CR) or a Complete Remission without an incomplete hematologic recovery (Cri)). In some embodiments, the improved treatment outcome is associated with an increase in IL-2 levels in the patient following administration of the expanded stem cell product. Increased IL-2 levels are an indication of increased immune response in the patient. Without intending to be bound by any particular theory, because the expanded stem cell product is derived from unmatched cord blood units from multiple human donors, the expanded hematopoietic stem cell product comprises hematopoietic stem cells or hematopoietic stem and progenitor cells having different HLA types and/or alleles. The presence of many mismatched HLA types or alleles of the expanded stem cell product after administration to the patient activates and/or increases the patient's immune response, potentially due to an increased antigen load. The resulting activation or stimulation of the patient's immune response may be due, in part, to activation of the patient's own T cells and/or NK cells.

The expanded stem cell product is not required or expected to engraft to provide therapeutic benefit to a patient. In some embodiments, the expanded stem cell product does not transiently or permanently engraft in the patient. In some embodiments, the expanded stem cell product does not transiently engraft in the patient. Engraftment is typically detected as mixed chimerism in the patient, meaning that cells from the expanded stem cell product are detected in the patient's blood about 7 to about 14 days after administration of the expanded stem cell product. In some embodiments, the expanded stem cell product does not measurably increase hematopoietic reconstitution, either transiently or long term. In some embodiments, the expanded stem cell product does not decrease the rate of infections in patients.

Frequent infections are a common complication of chemotherapy regimens used in the treatment of hematological malignancies, such as AML, and are a significant cause of treatment failure. Chemotherapy agents also can be profoundly immunosuppressive and/or highly myelosuppressive, which can lead to periods of prolonged neutropenia. Administration of the expanded stem cell product following a chemotherapy regimen can improve treatment outcome without necessarily preventing infectious complications or facilitating transient hematopoietic recovery post-chemotherapy, but rather by inducing a host immune response against the leukemia.

Preparation of an Expanded Stem Cell Product

The expanded stem cell product comprises hematopoietic stem or hematopoietic stem and progenitor cells and has been substantially depleted of T cells and red blood cells, therefore usually comprising enriched numbers of CD34+ hematopoietic stem or hematopoietic stem and progenitor cells. The hematopoietic stem or hematopoietic stem and progenitor cells comprise multiple HLA-types because the hematopoietic stem or hematopoietic stem and progenitor cells are not matched to each other prior to pooling and also are not matched to the patient. As used herein, substantially depleted of T cells refers to less than 1% CD3+ cells, or less than 0.5% CD3+ cells, or less than 0.1% CD3+ cells, in the expanded stem cell product.

In some embodiments, the CD34+ hematopoietic stem cells or hematopoietic stem and progenitor cells are derived from cord blood or from placental blood. Human umbilical cord blood and/or human placental blood are typical sources of the cord blood stem cells.

Such blood can be obtained by methods known in the art. See, e.g., U.S. Pat. Nos. 5,004,681 and 7,147,626 and U.S. Patent Publication No. 2013/0095079, incorporated herein by reference, for a discussion of collecting cord and placental blood at the birth of a human Umbilical cord blood and/or human placental blood collections are made under sterile conditions. Upon collection, cord or placental blood is mixed with an anticoagulant, such as CPD (citrate-phosphate-dextrose), ACD (acid citrate-dextrose), Alsever's solution (Alsever et al., 1941, N. Y. St. J. Med. 41:126), De Gowin's Solution (De Gowin, et al., 1940, J. Am. Med. Ass. 114:850), Edglugate-Mg (Smith, et al., 1959, J. Thorac. Cardiovasc. Surg. 38:573), Rous-Turner Solution (Rous and Turner, 1916, J. Exp. Med. 23:219), other glucose mixtures, heparin, ethyl biscoumacetate, and the like. See, generally, Hurn, 1968, Storage of Blood, Academic Press, New York, pp. 26-160). In one embodiment, ACD can be used.

Cord blood can preferably be obtained by direct drainage from the umbilical cord and/or by needle aspiration from the delivered placenta at the root and at distended veins. Preferably, the collected human cord blood and/or placental blood is free of contamination and, in particular, viral contamination.

In certain embodiments, the following tests can be performed on the collected blood, either routinely or where clinically indicated:

Bacterial culture: To ensure the absence of microbial contamination, established assays can be performed, such as routine hospital cultures for bacteria under aerobic and anaerobic conditions.

Diagnostic screening for pathogenic microorganisms: To ensure the absence of specific pathogenic microorganisms, various diagnostic tests can be employed. Diagnostic screening for any of the numerous pathogens transmissible through blood can be done by standard procedures. As one example, the collected blood sample (or a maternal blood sample) can be subjected to diagnostic screening for the presence of viruses. Any of numerous known assay systems can be used, based on the detection of virions, viral-encoded proteins, virus-specific nucleic acids, antibodies to viral proteins, and the like. The collected blood can also be tested for infectious diseases, including but not limited to Human Immunodeficiency Virus-1 or 2 (HIV-1 or HIV-2), human T-Cell lymphotropic virus I and II (HTLV-I and HTLV-II), Hepatitis B, Hepatitis C, Cytomegalovirus, Syphilis, corona virus, West Nile Virus, and the like.

Preferably, prior to collection of the cord blood, a maternal health history is determined to identify risks that the cord blood cells might pose, e.g., transmitting genetic or infectious diseases, such as cancer, leukemia, immune disorders, neurological disorders, hepatitis, or AIDS. The collected cord blood can have undergone testing for one or more of cell viability, HLA typing, ABO/Rh typing, CD34+ cell count, and total nucleated cell count.

Once the umbilical cord blood and/or placental blood is collected from human donors at birth, the blood is processed to produce an enriched hematopoietic stem cell population, or an enriched hematopoietic stem and progenitor cell population. Preferably, the hematopoietic stem cells, or hematopoietic stem and progenitor cells, are CD34+ cells or predominantly CD34+ cells. Preferably, the hematopoietic stem cell or hematopoietic stem and progenitor cell population is substantially depleted of T cells and of red blood cells, resulting in a cell population enriched CD34+ stem cells and/or CD34+ stem and progenitor cells. Enrichment thus refers to a process wherein the percentage of hematopoietic stem cells, or hematopoietic stem and progenitor cells, in the cell population is increased (relative to the percentage in the population before the enrichment procedure). Purification is one example of enrichment. In certain embodiments, the increase in the number of CD34+ cells (or other suitable antigen-positive cells) as a percentage of cells in the expanded stem cell product, relative to the population prior to the enrichment procedure, is at least 25-, 50-, 75-, 100-, 150-, 200-, 250-, 300-, 350-, 400- or at least 350-fold, and preferably is 100-200 fold or 100-400 fold.

Prior to processing for enrichment, the collected cord and/or placental blood can be fresh or a have been previously cryopreserved. Any suitable technique known in the art for cell separation/selection can be used to carry out the enrichment for hematopoietic stem cells, or hematopoietic stem and progenitor cells. Methods which rely on differential expression of cell surface markers can be used. For example, cells expressing the cell surface marker CD34 can be positively selected using a monoclonal antibody to CD34, such that cells expressing CD34 are retained, and cells not expressing CD34 are not retained. Moreover, the separation techniques employed should maximize the viability of the cell population to be selected. The particular technique employed will depend upon the efficiency of separation, cytotoxicity of the methodology, ease and speed of performance, and the necessity for sophisticated equipment and/or technical skill.

Procedures for separation may include magnetic separation, using antibody-coated magnetic beads, affinity chromatography, and “panning” with antibody attached to a solid matrix, e.g., plate, or other convenient technique. Techniques providing accurate separation/selection include fluorescence activated cell sorters, which can have varying degrees of sophistication, e.g., a plurality of color channels, low angle and obtuse light scattering detecting channels, impedance channels, and the like.

The antibodies used in the selection process may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Any technique may be employed which is not unduly detrimental to the viability of the remaining cells. Examples include, for example, the FDA approved CleniMACs® processing system (Miltenyl Biotec B.V. & Co. KG), the Dynabeads™ CD34 isolation system (Invtrogen Inc.), the EasySep™ Human CD34 Positive Selection Kit (Stemcell Technologies, Inc.), and the like.

In a preferred embodiment, fresh cord blood units are processed to select for, i.e., enrich for, CD34+ cells using anti-CD34 antibodies directly or indirectly conjugated to magnetic particles in connection with a magnetic cell separator, for example, the CliniMACS® Cell Separation System (Miltenyi Biotec, Bergisch Gladbach, Germany), which employs nano-sized super-paramagnetic particles composed of iron oxide and dextran coupled to specific monoclonal antibodies. The CliniMACS® Cell Separator is a closed sterile system, outfitted with a single-use disposable tubing set. The disposable tubing set can be used for and discarded after processing a single unit of collected cord and/or placental blood to enrich for CD34+ cells.

In an embodiment, two or more umbilical cord blood and/or placental blood units can be pooled prior to enriching for the hematopoietic stem cells, or hematopoietic stem and progenitor cells. In another embodiment, individual populations of CD34+ stem cells or CD34+ stem and progenitor cells can be pooled after enriching for the hematopoietic stem cells, or hematopoietic stem and progenitor cells. In specific embodiments, the number of umbilical cord blood and/or placental blood units, or populations of hematopoietic stem or hematopoietic stem and progenitor cells that are pooled is 2, 3, 4, 5, 6, 7, 8, 9, 10, 15, 20, 25, 30, 35, or 40, or at least any of the foregoing numbers. In some embodiments, the pool contains 2 to 8, 2 to 10, 4 to 8, 4 to 10, 2 to 20, 4 to 20, 2 to 25 or 4 to 25, and no more than 20 or 25, umbilical cord blood and/or placental blood units, or CD34+ hematopoietic stem or hematopoietic stem and progenitor cell populations. The umbilical cord blood and/or placental blood units or hematopoietic stem or hematopoietic stem and progenitor cell populations are pooled without regard to the HLA-type of the hematopoietic stem or hematopoietic stem and progenitor cells. In certain embodiments, the cells in the pool are derived from the umbilical cord blood and/or placental blood of individuals of the same race, e.g., African-American, Caucasian, Asian, Hispanic, Native-American, Australian Aboriginal, Inuit, Pacific Islander, or derived from umbilical cord blood and/or placental blood of individuals of the same ethnicity, e.g., Irish, Italian, Indian, Japanese, Chinese, Russian, and the like. In other embodiments, the cells in the pool are combined without regard to race or ethnicity.

Optionally, prior to enrichment for hematopoietic stem cells or hematopoietic stem and progenitor cells, the red blood cells and white blood cells of the cord blood or placental blood can be separated. Once the separation of the red blood cells and the white blood cells has taken place, the red blood cell fraction can be discarded, and the white blood cell fraction can be processed in the magnetic cell separator as described above to enrich for CD34+ hematopoietic stem cells or hematopoietic stem and progenitor cells. Separation of the white and red blood cell fractions can be performed by any method known in the art, including centrifugation techniques. Other separation methods that can be used include, for example, the use of commercially available products FICOLL™ or FICOLL-PAQUE™ or PERCOLL™ (GE Healthcare, Piscataway, N.J.). FICOLL-PAQUE™ is normally placed at the bottom of a conical tube, and the whole blood is layered above. After being centrifuged, the following layers will be visible in the conical tube, from top to bottom: plasma and other constituents, a layer of mono-nuclear cells called buffy coat containing the peripheral blood mononuclear cells (white blood cells), FICOLL-PAQUE™, and erythrocytes and granulocytes, which should be present in pellet form. This separation technique allows easy harvest of the peripheral blood mononuclear cells (PBMCs).

Optionally, prior to CD34+ cell selection, an aliquot of the cord blood or placental unit can be checked for total nucleated cell count and/or CD34+ cell content. In a specific embodiment, after the CD34+ cell selection, both CD34+ and CD34− cell fractions are recovered. Optionally, DNA can be extracted from a sample of the CD34− cell fraction for initial HLA typing and future chimerism studies, even though HLA matching of the CD34+ cell fraction to the patient or to the other cord blood or placental blood units is not done. The CD34+ enriched stem cell or stem and progenitor cell population can be subsequently processed prior to expansion, for example, by suspension in an appropriate cell culture medium for storage or transport. In a preferred embodiment, the cell culture medium is a cell culture medium suitable for the maintenance of viability of CD34+ hematopoietic stem cell or hematopoietic stem and progenitor cells. For example, the cell culture medium can be STEMSPAN™ Serum Free Expansion Medium or STEMSPAN™ Serum Free Expansion Medium II (StemCell Technologies, Vancouver, British Columbia) in the presence of growth factors, for example, present at the following concentrations: 50-300 ng/ml of stem cell factor (SCF), 50-300 ng/ml of Flt-3 receptor ligand (Flt3L), 50-100 ng/ml of Thrombopoietin (TPO), 50-100 ng/ml of Interleukin-6 (IL-6), and 10 ng/ml of Interleukin-3 (IL-3). In more specific embodiments, 300 ng/ml of stem cell factor, 300 ng/ml of Flt-3 receptor ligand, 100 ng/ml of Thrombopoietin, 100 ng/ml of Interleukin-6 and 10 ng/ml of Interleukin-3, or 50 ng/ml of stem cell factor, 50 ng/ml of Flt-3 receptor ligand, 50 ng/ml of Thrombopoietin, 50 ng/ml of Interleukin-6 and 10 ng/ml of Interleukin-3, are used. In another preferred embodiment, the cell culture medium consists of STEMSPAN™ Serum Free Expansion Medium or STEMSPAN™ Serum Free Expansion Medium II (StemCell Technologies, Vancouver, British Columbia) supplemented with 10 ng/ml recombinant human Interleukin-3 (rhIL-3), 50 ng/ml recombinant human Interleukin-6 (rhIL-6), 50 ng/ml recombinant human Thrombopoietin (rhTPO), 50 ng/ml recombinant human Flt-3 Ligand (rhFlt-3L), 50 ng/ml and recombinant human stem cell factor (rhSCF). In another preferred embodiment, the cell culture medium consists of StemSpan Serum Free Expansion Medium II (SFEM II, StemCell Technologies, Vancouver, British Columbia) supplemented with recombinant human rhSCF, rhFlt-3L, rhTPO, rhIL-6 (each at 50 ng/ml final concentration), and rhIL-3 (at 10 ng/ml final concentration).

In a specific embodiment, the umbilical cord blood and/or placental blood units are red cell depleted, and the number of CD34+ cells in the red cell depleted fraction is determined. Preferably, the umbilical cord blood and/or placental blood samples containing more than 3.5 million CD34+ cells are subject to the enrichment methods described above.

After the hematopoietic stem cells or hematopoietic stem and progenitor cells have been isolated (e.g., from human cord blood and/or human placental blood collected from humans at birth) according to the enrichment methods described above or other methods known in the art, the hematopoietic stem cells or hematopoietic stem and progenitor cells are expanded to increase the number of hematopoietic stem cells or hematopoietic stem and progenitor cells, e.g., CD34+ cells. Any method known in the art for expanding the number of hematopoietic stem cells or hematopoietic stem and progenitor cells that gives rise to an expanded (i.e., increased number of) population of hematopoietic stem cells or hematopoietic stem and progenitor cells can be used. Preferably, the hematopoietic stem cells or hematopoietic stem and progenitor cells are cultured under cell growth conditions (e.g., promoting mitosis) such that the hematopoietic stem cells or hematopoietic stem and progenitor cells grow and divide (proliferate) to obtain an expanded population of CD34+ hematopoietic stem cells or hematopoietic stem and progenitor cells. In one embodiment, individual populations of hematopoietic stem cells or hematopoietic stem and progenitor cells derived from an umbilical cord blood and/or placental blood of a single human at birth can be pooled, without matching to the HLA type of the other hematopoietic stem cells or hematopoietic stem and progenitor cells, prior to or after expansion. In another embodiment, the hematopoietic stem cells or hematopoietic stem and progenitor cells are expanded prior to pooling. Preferably, the technique used for expansion is one that has been shown to (i) result in an increase in the number of hematopoietic stem cells, or hematopoietic stem and progenitor cells, e.g., CD34+ cells, in the expanded stem cell product relative to the unexpanded population of hematopoietic stem cells or stem and progenitor cells, where the unexpanded cell population and expanded cell population are from different aliquots of the same source of stem or stem and progenitor cells, wherein the expanded cells but not the unexpanded cells are subjected to the expansion technique.

Expansion techniques include, but are not limited to those described in U.S. Pat. No. 7,399,633 B2; U.S. Patent Publication No. 2013/0095079; Delaney et al., 2010, Nature Med. 16(2): 232-236; Zhang et al., 2008, Blood 111:3415-3423; or Himburg et al., 2010, Nature Medicine 16(4):475-82, each incorporated herein by reference, as well as those described below.

In one embodiment, the hematopoietic stems cells or hematopoietic stem and progenitor cells are cultured in culture medium in the presence of growth factors, and are exposed to cell growth conditions (e.g., promoting mitosis) such that the hematopoietic stem or hematopoietic stem and progenitor cells proliferate to obtain an expanded population of hematopoietic stem or hematopoietic stem and progenitor cells. In a preferred embodiment, the hematopoietic stem or hematopoietic stem and progenitor cells are cultured in the presence of an amount of an agonist of Notch function effective to inhibit differentiation (typically an immobilized agonist of Notch function), and are exposed to cell growth conditions (e.g., promoting mitosis) such that the hematopoietic stem or hematopoietic stem and progenitor cells proliferate to generate an expanded hematopoietic stem or hematopoietic stem and progenitor cell population. In a more preferred embodiment, the hematopoietic stem or hematopoietic stem and progenitor cells are cultured with an amount of an agonist of Notch function effective to inhibit differentiation and in the presence of growth factors, and are exposed to cell growth conditions (e.g., promoting mitosis) such that the hematopoietic stem or hematopoietic stem and progenitor cells proliferate to obtain an expanded hematopoietic stem or hematopoietic stem and progenitor cell population. The expanded hematopoietic stem or hematopoietic stem and progenitor cell population so obtained can be frozen and stored for later use, as an “off-the-shelf product”. Optionally, the Notch pathway agonist is inactivated or removed from the expanded hematopoietic stem or hematopoietic stem and progenitor cell population prior to transplantation into the patient (e.g., by separation or dilution).

In specific embodiments, the hematopoietic stem or hematopoietic stem and progenitor cells are cultured for 2, 3, 4, 5, 6, 7, 8, 9, 10, 11, 12, 13, 14, 15, 16, 17, 18, 19, 20, 21, 22, 23, 24, or 25 days or more; or, preferably, the hematopoietic stem or hematopoietic stem and progenitor cells are cultured for at least 10 days or from about 7 to about 14 days.

An exemplary culture condition for expanding the hematopoietic stem or hematopoietic stem and progenitor cells includes culturing the cells for 7 to 14 days in the presence of fibronectin fragments and the extracellular domain of a Delta protein fused to the Fc domain of human IgG (Delta1^{ext-IgG}) in serum free medium supplemented with the following human growth factors: stem cell factor, Flt-3 receptor ligand, Thrombopoietin, Interleukin-6 and Interleukin-3. Preferably, the foregoing growth factors are present at the following concentrations: 50-300 ng/ml stem cell factor, 50-300 ng/ml Flt-3 receptor ligand, 50-100 ng/ml Thrombopoietin, 50-100 ng/ml Interleukin-6 and 10 ng/ml Interleukin-3. In more specific embodiments, 300 ng/ml stem cell factor, 300 ng/ml of Flt-3 receptor ligand, 100 ng/ml Thrombopoietin, 100 ng/ml Interleukin-6 and 10 ng/ml Interleukin-3, or 50 ng/ml stem cell factor, 50 ng/ml of Flt-3 receptor ligand, 50 ng/ml Thrombopoietin, 50 ng/ml Interleukin-6 and 10 ng/ml Interleukin-3, are used. In a more preferred embodiment, the cell culture medium consists of STEMSPAN™ Serum Free Expansion Medium (StemCell Technologies, Vancouver, British Columbia) supplemented with 10 ng/ml recombinant human Interleukin-3 (rhIL-3), 50 ng/ml recombinant human Interleukin-6 (rhIL-6), 50 ng/ml recombinant human Thrombopoietin (rhTPO), 50 ng/ml recombinant human Flt-3 Ligand (rhFlt-3L), 50 ng/ml and recombinant human stem cell factor (rhSCF). In another more preferred embodiment, the cell culture medium consists of StemSpan Serum Free Expansion Medium II (SFEM II, StemCell Technologies, Vancouver, British Columbia) supplemented with recombinant human rhSCF, rhFlt-3L, rhTPO, rhIL-6 (each at 50 ng/ml final concentration), and rhIL-3 (at 10 ng/ml final concentration).

In some embodiments, DXI-mediated expansion is performed as follows: Delta1^{ext-IgG} (DXI) is immobilized on the surface of the cell culture dishes. In a specific embodiment, the cell culture dishes are coated overnight at 4° C. (or for a minimum of 2 hours at 37° C.) with 2.5 μg/ml Delta1^{ext-IgG} and 5 μg/ml RetroNectin® (a recombinant human fibronectin fragment also referred to as rFN-CH-296) in phosphate buffered saline, before adding the hematopoietic stem or hematopoietic stem and progenitor cells. Preferably the cell culture medium consists of STEMSPAN™ Serum Free Expansion Medium (StemCell Technologies, Vancouver, British Columbia) supplemented with 10 ng/ml recombinant human Interleukin-3 (rhIL-3), 50 ng/ml recombinant human Interleukin-6 (rhIL-6), 50 ng/ml recombinant human Thrombopoietin (rhTPO), 50 ng/ml recombinant human Flt-3 Ligand (rhFlt-3L), 50 ng/ml and recombinant human stem cell factor (rhSCF), or StemSpan Serum Free Expansion Medium II (SFEM II, StemCell Technologies, Vancouver, British Columbia) supplemented with recombinant human rhSCF, rhFlt-3L, rhTPO, rhIL-6 (each at 50 ng/ml final concentration), and rhIL-3 (at 10 ng/ml final concentration).

Other exemplary culture conditions for expanding hematopoietic stem or stem and progenitor cells are set forth in Zhang et al., 2008, Blood 111:3415-3423, (incorporated herein by reference). In a specific embodiment, the hematopoietic stem or hematopoietic stem and progenitor cells can be cultured in serum free medium supplemented with heparin, stem cell factor, Thrombopoietin, insulin-like growth factor-2 (IGF-2), fibroblast growth factor-1 (FGF-1), and Angptl3 or Angptl5. In a specific embodiment, the medium is supplemented with 10 μg/ml heparin, 10 ng/ml stem cell factor, 20 ng/ml Thrombopoietin, 20 ng/ml IGF-2, and 10 ng/ml FGF-1, and 100 ng/ml Angptl3 or Angptl5 and the cells are cultured for about 19 to 23 days. In another specific embodiment, the hematopoietic stem or hematopoietic stem and progenitor cells can be expanded by culturing the cells in serum free medium supplemented with 10 μg/ml heparin, 10 ng/ml stem cell factor, 20 ng/ml Thrombopoietin, 10 ng/ml FGF-1, and 100 ng/ml Angptl5 for about 11 to 19 days. In another specific embodiment, the hematopoietic stem or stem and progenitor cells can be expanded by culturing the cells in serum free medium supplemented with 50 ng/ml stem cell factor, 10 ng/ml Thrombopoietin, 50 ng/ml Flt-3 receptor ligand, and 100 ng/ml insulin-like growth factor binding protein-2 (IGFBP2) or 500 ng/ml Angptl5 for about 10 days. In yet another embodiment, the hematopoietic stem or hematopoietic stem and progenitor cells can be expanded by culturing the cells in serum free medium supplemented with 10 μg/ml heparin, 10 ng/ml stem cell factor, 20 ng/ml Thrombopoietin, 10 ng/ml FGF-1, 500 ng/ml Angptl5, and 500 ng/ml IGFBP2 for about 11 days. See Zhang et al., 2008, Blood 111:3415-3423, incorporated herein by reference.

Another exemplary culture condition for expanding the hematopoietic stem or hematopoietic stem and progenitor cells is set forth in Himburg et al., 2010, Nature Medicine 16(4):475-482, incorporated herein by reference. In a specific embodiment, the hematopoietic stem or hematopoietic stem and progenitor cells can be cultured in liquid suspension culture supplemented with Thrombopoietin, stem cell factor, Flt-3 receptor ligand, and pleiotrophin. In a specific embodiment, the liquid suspension culture is supplemented with 20 ng/ml Thrombopoietin, 125 ng/ml stem cell factor, 50 ng/ml Flt-3 receptor ligand, and 10, 100, 500, or 1000 ng/ml pleiotrophin and the hematopoietic stem or hematopoietic stem and progenitor cells are cultured for about 7 days.

After expansion of the hematopoietic stem or hematopoietic stem and progenitor cells, the total number of cells and viable CD34+ cells are determined. For example, at day 14 during expansion, a sample can be taken for determination of the total viable nucleated cell count. In addition, the total number of CD34+ cells can be determined by multi-parameter flow cytometry, and, thus, the percentage of CD34+ cells in the sample. Preferably, cultures that have not resulted in at least a 10-fold increase in the absolute number of CD34+ cells are discontinued. Similarly, prior to cryopreservation or after thawing, an aliquot of the expanded hematopoietic stem or hematopoietic stem and progenitor cell population can be taken for determination of total nucleated cells and percentage of viable CD34+ cells in order to calculate the total viable CD34+ cell number in the expanded population. In a preferred embodiment, those populations containing less than 50 million CD34+ viable cells can be discarded.

In a specific embodiment, total viable CD34+(or other antigen-positive) cell numbers can be considered the potency assay for release of the final product for therapeutic use. Viability can be determined by any method known in the art, for example, by trypan blue exclusion or 7-amino-actinomycin D (7-AAD) exclusion. Preferably, the total nucleated cell count (TNC) and other data are used to calculate the potency of the product. The percentage of viable CD34+ cells can be assessed by flow cytometry and use of a stain that is excluded by viable cells. The percentage of viable CD34+ cells=the number of CD34+ cells that exclude 7-AAD (or other appropriate stain) in an aliquot of the sample divided by the TNC (both viable and non-viable) of the aliquot. Viable CD34+ cells in the sample can be calculated as follows: Viable CD34+ cells=TNC of sample x % viable CD34+ cells in the sample. The proportional increase during enrichment or expansion in viable CD34+ cells can be calculated as follows: Total Viable CD34+ cells Post-culture/Total Viable CD34+ cells Pre-culture.

In some embodiments, the hematopoietic stem or hematopoietic stem and progenitor cells are expanded by culturing the cells in the presence of an agonist of Notch function and one of more growth factors or cytokines for a given period of time, as described above. An agonist of Notch function, also referred to as Notch agonist, is an agent that promotes, i.e., causes or increases, activation of Notch pathway function. As used herein, “Notch function” means a function mediated by the Notch signaling (signal transduction) pathway, including but not limited to nuclear translocation of the intracellular domain of Notch, nuclear translocation of RBP-R or its Drosophila homolog Suppressor of Hairless; activation of bHLH genes of the Enhancer of Split complex, e.g., Mastermind; activation of the HES-1 gene or the KBF2 (also called CBF1) gene; inhibition of Drosophila neuroblast segregation; and binding of Notch to Delta, Jagged/Serrate, Fringe, Deltex or RBP-Jκ/Suppressor of Hairless, or homologs or analogs thereof. See generally the review article by Kopan et al., 2009, Cell 137:216-233 for a discussion of the Notch signal transduction pathway and its effects upon activation; see also Jarriault et al., 1998, Mol. Cell. Biol. 18:7423-7431, both incorporated herein by reference in their entirety.

Notch activation is carried out by exposing a cell to a Notch agonist. The agonist of Notch function can be but is not limited to a soluble molecule, a molecule that is recombinantly expressed on a cell-surface, a molecule on a cell monolayer to which the hematopoietic stem or hematopoietic stem and precursor cells are exposed, or a molecule immobilized on a solid phase. Exemplary Notch agonists are the extracellular binding ligands Delta and Serrate which bind to the extracellular domain of Notch and activate Notch signal transduction, or a fragment of Delta or Serrate that binds to the extracellular domain of Notch and activates Notch signal transduction. Nucleic acid and amino acid sequences of Delta and Serrate have been isolated from several species, including human, are known in the art, and are disclosed in International Patent Publication Nos. WO 93/12141, WO 96/27610, WO 97/01571, and Gray et al., 1999, Am. J. Path. 154:785-794. In a preferred embodiment, the Notch agonist is an immobilized fragment of a Delta or Serrate protein consisting of the extracellular domain of the protein fused to a myc epitope tag (Delta^{ext-myc} or Serrate^{ext-myc}, respectively) or an immobilized fragment of a Delta or Serrate protein consisting of the extracellular domain of the protein fused to the Fc portion of IgG (Delta or Serrate respectively). Notch agonists include but are not limited to Notch proteins and analogs and derivatives (including fragments) thereof; proteins that are other elements of the Notch pathway and analogs and derivatives (including fragments) thereof; antibodies thereto and fragments or other derivatives of such antibodies containing the binding region thereof; nucleic acids encoding the proteins and derivatives or analogs; as well as proteins and derivatives and analogs thereof which bind to or otherwise interact with Notch proteins or other proteins in the Notch pathway such that Notch pathway activity is promoted. Such agonists include but are not limited to Notch proteins and derivatives thereof comprising the intracellular domain, Notch nucleic acids encoding the foregoing, and proteins comprising the Notch-interacting domain of Notch ligands (e.g., the extracellular domain of Delta or Serrate). Other agonists include but are not limited to RBPR/Suppressor of Hairless or Deltex. Fringe can be used to enhance Notch activity, for example in conjunction with Delta protein. These proteins, fragments and derivatives thereof can be recombinantly expressed and isolated or can be chemically synthesized.

In another specific embodiment, the Notch agonist is a cell which recombinantly expresses a protein or fragment or derivative thereof, which agonizes Notch. The cell expresses the Notch agonist in such a manner that it is made available to the hematopoietic stem cells or stem and progenitor cells in which Notch signal transduction is to be activated, e.g., it is secreted, expressed on the cell surface, etc.

In yet another specific embodiment, the agonist of Notch is a peptidomimetic or peptide analog or organic molecule that binds to a member of the Notch signaling pathway. Such an agonist can be identified by binding assays selected from those known in the art, for example the cell aggregation assays described in Rebay et al., 1991, Cell 67:687-699 and in International Patent Publication No. WO 92/19734, both incorporated herein by reference.

In a preferred embodiment the agonist is a protein consisting of at least a fragment of a protein encoded by a Notch-interacting gene which mediates binding to a Notch protein or a fragment of Notch, which fragment of Notch contains the region of Notch responsible for binding to the agonist protein, e.g., epidermal growth factor-like repeats 11 and 12 of Notch. Notch interacting genes, as used herein, shall mean the genes Notch, Delta, Serrate, RBPJκ, Suppressor of Hairless and Deltex, as well as other members of the Delta/Serrate family or Deltex family which may be identified by virtue of sequence homology or genetic interaction and more generally, members of the “Notch cascade” or the “Notch group” of genes, which are identified by molecular interactions (e.g., binding in vitro, or genetic interactions (as depicted phenotypically, e.g., in Drosophila). Exemplary fragments of Notch-binding proteins containing the region responsible for binding to Notch are described in U.S. Pat. Nos. 5,648,464; 5,849,869; and 5,856,441, incorporated herein by reference.

The Notch agonists utilized by the methods described herein can be obtained commercially, produced by recombinant expression, or chemically synthesized.

In a specific embodiment, exposure of the cells to a Notch agonist is not done by incubation with other cells recombinantly expressing a Notch ligand on the cell surface (although in other embodiments, this method can be used), but rather is by exposure to a cell-free Notch ligand, e.g., incubation with a cell-free ligand of Notch, which ligand is immobilized on the surface of a solid phase, e.g., immobilized on the surface of a tissue culture substrate, a dish, flask, bottle, bag, and the like.

In specific embodiments, Notch activity is promoted by the binding of Notch ligands (e.g., Delta, Serrate) to the extracellular portion of the Notch receptor. Notch signaling appears to be triggered by the physical interaction between the extracellular domains of Notch and its ligands that are either membrane-bound on adjacent cells or immobilized on a solid surface. Full length ligands are agonists of Notch, as their expression on one cell triggers the activation of the pathway in the neighboring cell which expresses the Notch receptor. Soluble truncated Delta or Serrate molecules, comprising the extracellular domains of the proteins or Notch-binding portions thereof, that have been immobilized on a solid surface, such as a tissue culture plate, are particularly preferred Notch pathway agonists. Such soluble proteins can be immobilized on a solid surface by an antibody or interacting protein, for example an antibody directed to an epitope tag with which Delta or Serrate is expressed as a fusion protein (e.g., a myc epitope tag, which is recognized by the antibody 9E10) or a protein which interacts with an epitope tag with which Delta or Serrate is expressed as a fusion protein (e.g., an immunoglobulin epitope tag, which is bound by Protein A).

In another specific embodiment, and as described in U.S. Pat. No. 5,780,300 to Artavanis-Tsakonas et al., Notch agonists include reagents that promote or activate cellular processes that mediate the maturation or processing steps required for the activation of Notch or a member of the Notch signaling pathway, such as the furin-like convertase required for Notch processing, Kuzbanian, the metalloprotease-disintegrin (ADAM) thought to be required for the activation of the Notch pathway upstream or parallel to Notch (Schlondorff and Blobel, 1999, J. Cell Sci. 112:3603-3617), or, more generally, cellular trafficking and processing proteins such as the rab family of GTPases required for movement between cellular compartments (for a review on Rab GTPases, see Olkkonen and Stenmark, 1997, Int. Rev. Cytol. 176:1-85). The agonist can be any molecule that increases the activity of one of the above processes, such as a nucleic acid encoding a furin, Kuzbanian or rab protein, or a fragment or derivative or dominant active mutant thereof, or a peptidomimetic or peptide analog or organic molecule that binds to and activates the function of the above proteins.

U.S. Pat. No. 5,780,300 (incorporated herein by reference) further discloses classes of Notch agonist molecules (and methods of their identification) which can be used to activate the Notch pathway, for example molecules that trigger the dissociation of the Notch ankyrin repeats with RBP-R, thereby promoting the translocation of RBP-R from the cytoplasm to the nucleus.

In some preferred embodiments, a DXI expansion method is used. The Notch agonist is an immobilized fragment of a Delta consisting of the extracellular domain of the protein fused to the Fc portion of IgG (Delta^{ext-IgG} or DXI), as described in U.S. Pat. No. 7,399,633 or an immobilized Notch-1 or Notch-2 specific antibody, as described in U.S. Pat. No. 10,208,286 (both incorporated herein by reference). Preferably, Delta1^{ext-IgG} is immobilized on the surface of a cell culture dish. In a specific embodiment, cell culture dishes are coated overnight at 4° C. (or for a minimum of 2 hours at 37° C.) with 2.5 μg/ml Delta1^{ext-IgG} and 5 μg/ml RetroNectin® (a recombinant human fibronectin fragment also referred to as rFN-CH-296) in phosphate buffered saline, before adding the hematopoietic stem or hematopoietic stem and progenitor cells. Preferably, the cell culture medium consists of StemSpan™ Serum Free Expansion Medium (StemCell Technologies, Vancouver, British Columbia) supplemented with 10 ng/ml recombinant human Interleukin-3 (rhIL-3), 50 ng/ml recombinant human Interleukin-6 (rhIL-6), 50 ng/ml recombinant human Thrombopoietin (rhTPO), 50 ng/ml recombinant human Flt-3 Ligand (rhFlt-3L), 50 ng/ml and recombinant human stem cell factor (rhSCF), or StemSpan Serum Free Expansion Medium II (SFEM II, StemCell Technologies, Vancouver, British Columbia) supplemented with recombinant human rhSCF, rhFlt-3L, rhTPO, rhIL-6 (each at 50 ng/ml final concentration), and rhIL-3 (at 10 ng/ml final concentration). The hematopoietic stem or hematopoietic stem and progenitor cells are cultured for about 7 to about 14 days.

Once the expanded hematopoietic stem cells or hematopoietic stem and progenitor cells are obtained to form the expanded stem cell product, the expanded hematopoietic stem or hematopoietic stem and progenitor cell population can be collected and cryopreserved, e.g., to prepare an “off-the-shelf” product. In one embodiment, an expanded hematopoietic stem cell or hematopoietic stem and progenitor cell population can be divided and frozen in one or more bags (or units). In another embodiment, two or more expanded hematopoietic stem cell or hematopoietic stem and progenitor cell populations can be pooled, divided into separate aliquots, and each aliquot is frozen. In a preferred embodiment, from about 50 to about 400 million CD34+ cells are frozen in a single bag (or unit) of expanded stem cell product. In another preferred embodiment, from about 100 to about 300 million CD34+ cells are frozen in a single bag (or unit) of expanded stem cell product. In other preferred embodiments, about 100, 200, 300 or 400 million CD34+ cells are frozen in a single bag (or unit) of expanded stem cell product.

In a preferred embodiment, the expanded stem product is fresh, i.e., it has not been previously frozen prior to expansion or cryopreservation. The terms “frozen/freezing” and “cryopreserved/cryopreserving” are used interchangeably in the present application. Cryopreservation can be by any method known in the art that freezes cells in viable form. The freezing of cells is ordinarily destructive. On cooling, water within the cell freezes. Injury then occurs by osmotic effects on the cell membrane, cell dehydration, solute concentration, and ice crystal formation. As ice forms outside the cell, available water is removed from solution and withdrawn from the cell, causing osmotic dehydration and raised solute concentration which eventually destroy the cell. For a discussion, see Mazur, P., 1977, Cryobiology 14:251-272.

These injurious effects can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO) (Lovelock and Bishop, 1959, Nature 183:1394-1395; Ashwood-Smith, 1961, Nature 190:1204-1205), glycerol, polyvinylpyrrolidine (Rinfret, 1960, Ann. N.Y. Acad. Sci. 85:576), polyethylene glycol (Sloviter and Ravdin, 1962, Nature 196:548), albumin, dextran, sucrose, ethylene glycol, i-erythritol, D-ribitol, D-mannitol (Rowe et al., 1962, Fed. Proc. 21:157), D-sorbitol, i-inositol, D-lactose, choline chloride (Bender et al., 1960, J. Appl. Physiol. 15:520), amino acids (Phan The Tran and Bender, 1960, Exp. Cell Res. 20:651), methanol, acetamide, glycerol monoacetate (Lovelock, 1954, Biochem. J. 56:265), inorganic salts (Phan The Tran and Bender, 1960, Proc. Soc. Exp. Biol. Med. 104:388; Phan The Tran and Bender, 1961, in Radiobiology, Proceedings of the Third Australian Conference on Radiobiology, Ilbery ed., Butterworth, London, p. 59), and CryoStor® CS10 (BioLife Solutions Inc., Bothell, Wash.). In a preferred embodiment, DMSO is used, a liquid which is nontoxic to cells in low concentration. Addition of plasma (e.g., to a concentration of about 20-25%) can augment the protective effect of DMSO. After addition of DMSO, cells should be kept at 0° C. until freezing, since DMSO concentrations of about 1% are toxic at temperatures above 4° C.

A controlled slow cooling rate can be important. Different cryoprotective agents (Rapatz et al., 1968, Cryobiology 5(1):18-25) and different cell types have different optimal cooling rates (see e.g., Rowe and Rinfret, 1962, Blood 20:636; Rowe, 1966, Cryobiology 3(1):12-18; Lewis, et al., 1967, Transfusion 7(1):17-32; and Mazur, 1970, Science 168:939-949 for effects of cooling velocity on survival of marrow-stem cells and on their transplantation potential). The heat of fusion phase where water turns to ice should be minimal. The cooling procedure can be carried out by use of, e.g., a programmable freezing device or a methanol bath procedure.

A programmable freezing apparatus allows for the determination of optimal cooling rates and facilitates standard reproducible cooling. Programmable controlled-rate freezers such as Cryomed or Planar permit tuning of the freezing regimen to the desired cooling rate curve. For example, for marrow cells in 10% DMSO and 20% plasma, the optimal rate is 1° to 3° C./minute from 0° C. to −80° C. In a preferred embodiment, this cooling rate can be used. The container holding the cells must be stable at cryogenic temperatures and allow for rapid heat transfer for effective control of both freezing and thawing. Sealed plastic vials (e.g., Nunc, Wheaton cryules) or glass ampules can be used for multiple small amounts (1-2 ml), while larger volumes (100-200 ml) can be frozen in polyolefin bags (e.g., Delmed) held between metal plates for better heat transfer during cooling. Bags of bone marrow cells have been successfully frozen by placing them in −80° C. freezers which, fortuitously, gives a cooling rate of approximately 3° C./minute).

In an alternative embodiment, the methanol bath method of cooling can be used. The methanol bath method is well-suited to routine cryopreservation of multiple small items on a large scale. The method does not require manual control of the freezing rate nor a recorder to monitor the rate. In a preferred embodiment, DMSO-treated cells are pre-cooled on ice and transferred to a tray containing chilled methanol which is placed, in turn, in a mechanical refrigerator (e.g., Harris or Revco) at −80° C. Thermocouple measurements of the methanol bath and the samples indicate the desired cooling rate of 1° to 3° C./minute. After at least two hours, the specimens have reached a temperature of −80° C. and can be placed directly into liquid nitrogen (−196° C.) for permanent storage.

After thorough freezing, the expanded stem cell product can be rapidly transferred to a long-term cryogenic storage vessel. In a preferred embodiment, samples can be cryogenically stored in liquid nitrogen (−196° C.) or its vapor (−165° C.). Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum.

Suitable racking systems are commercially available and can be used for cataloguing, storage, and retrieval of individual specimens.

Considerations and procedures for the manipulation, cryopreservation, and long-term storage of the hematopoietic stem cells, particularly from bone marrow or peripheral blood, are largely applicable to expanded hematopoietic stem cells or stem and progenitor cells. Such a discussion can be found, for example, in the following references, incorporated by reference herein: Gorin, 1986, Clinics In Haematology 15(1):19-48; Bone-Marrow Conservation, Culture and Transplantation, Proceedings of a Panel, Moscow, Jul. 22-26, 1968, International Atomic Energy Agency, Vienna, pp. 107-186.

Other methods of cryopreservation of viable cells, or modifications thereof, are available and envisioned for use (e.g., cold metal-mirror techniques; Livesey and Linner, 1987, Nature 327:255; Linner et al., 1986, J. Histochem. Cytochem. 34(9):1123-1135; see also U.S. Pat. No. 4,199,022 by Senkan et al., U.S. Pat. No. 3,753,357 by Schwartz, U.S. Pat. No. 4,559,298 by Fahy).

Cryopreserved or frozen cells are preferably thawed quickly (e.g., in a water bath maintained at 37°−41° C.) and chilled immediately upon thawing. In a specific embodiment, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

In an embodiment of the invention, the expanded stem cell product is thawed, or a portion thereof, can be infused in a human patient in need thereof (e.g., having AML or other hematological malignancy. Several procedures, relating to processing of the thawed cells, are available and can be employed if deemed desirable.

It may be desirable to treat the cells in order to prevent cellular clumping upon thawing. To prevent clumping, various procedures can be used, including but not limited to, the addition before and/or after freezing of DNase (Spitzer et al., 1980, Cancer 45:3075-3085), low molecular weight dextran and citrate, hydroxyethyl starch (Stiff et al., 1983, Cryobiology 20:17-24), etc.

The cryoprotective agent, if toxic in humans, should be removed prior to therapeutic use of the thawed expanded stem cell product. In an embodiment employing DMSO as the cryopreservative, it is preferable to omit this step to avoid cell loss. However, where removal of the cryoprotective agent is desired, the removal is preferably accomplished upon thawing.

One way in which to remove the cryoprotective agent is by dilution to an insignificant concentration. This can be accomplished by addition of medium, followed by, if necessary, one or more cycles of centrifugation to pellet cells, removal of the supernatant, and resuspension of the cells. For example, intracellular DMSO in the thawed cells can be reduced to a level (less than 1%) that will not adversely affect the recovered cells. This is preferably done slowly to minimize potentially damaging osmotic gradients that occur during DMSO removal.

After removal of the cryoprotective agent, cell count (e.g., by use of a hemocytometer) and viability testing (e.g., by trypan blue exclusion; Kuchler, 1977, Biochemical Methods in Cell Culture and Virology, Dowden, Hutchinson & Ross, Stroudsburg, Pa., pp. 18-19; 1964, Methods in Medical Research, Eisen et al., eds., Vol. 10, Year Book Medical Publishers, Inc., Chicago, pp. 39-47) can be done to confirm cell survival. The percentage of viable antigen (e.g., CD34) positive cells can be determined by calculating the number of antigen positive cells that exclude 7-AAD (or other suitable dye excluded by viable cells) in an aliquot of the cells, divided by the total number of nucleated cells (TNC) (both viable and non-viable) in the aliquot of the cells. The number of viable antigen positive cells can be then determined by multiplying the percentage of viable antigen positive cells by the TNC.

Prior to cryopreservation and/or after thawing, the total number of nucleated cells, or in a specific embodiment, the total number of CD34+ cells can be determined. For example, total nucleated cell count can be performed by using a hemocytometer and exclusion of trypan blue dye. Specimens that are of high cellularity can be diluted to a concentration range appropriate for manual counting. Final cell counts for products are corrected for any dilution factors. Total nucleated cell count=viable nucleated cells per mL×volume of product in mL. The number of CD34+ positive cells in the sample can be determined, e.g., by use of flow cytometry using anti-CD34 monoclonal antibodies conjugated to a fluorochrome.

In certain embodiments, the identity and purity of the starting hematopoietic stem cell or stem and progenitor cell population, umbilical cord blood and/or placental blood, or the expanded stem cell product prior to cryopreservation, or the expanded stem cell product after thawing can be subjected to multi-parameter flow cytometric immunophenotyping, which provides the percentage of viable antigen positive cells present in a sample. Each sample can be tested for one or more of the following cell phenotypes using a panel of monoclonal antibodies directly conjugated to fluorochromes:

1. CD34+ HPC

2. T cells (CD3+, including both CD4+ and CD8+ subsets)

3. B cells (CD19+ or CD20+)

4. NK cells (CD56+)

5. Monocytes (CD14+)

6. Myelomonocytes (CD15+)

7. Megakaryocytes (CD41+)

8. Dendritic Cells (lineage negative/HLA-DRbright and CD123bright, or lineage negative/HLA-DRbright and CD11cbright).

Therapeutic Methods

In accordance with the present invention, methods for improving a treatment outcome for a patient having AML or other hematological malignancy are provided. Methods for treating a patient having AML or other hematological malignancy are also provided. The patient is treated by administering a chemotherapy regimen, or a cycle thereof, and then administering a fixed dose of an expanded stem cell product to the patient, wherein the administering is done without matching the HLA-type of the expanded stem cell product to the HLA-type of the patient. The expanded stem cell product is a pooled product derived from the hematopoietic stem or hematopoietic stem and progenitor cells from at least two or at least four human donors without matching to the HLA types of the donors to each other and also without matching to the HLA type of the patient. The phrase “without matching the HLA-type,” means no steps are taken to have any of the HLA antigens or alleles match between the patient and/or between the donors contributing to the expanded stem cell product (or the hematopoietic stem or hematopoietic stem and progenitor cells in the expanded stem cell product).

In some embodiments, a fixed dose of the expanded stem cell product can be administered following a chemotherapy regimen or a cycle thereof, such as an induction regimen. A fixed dose of the expanded stem cell product can also be administered following a consolidation regimen or a cycle thereof. A fixed dose of the expanded stem cell product can also be administered following a salvage regimen or a cycle thereof. In some embodiments, a fixed dose of the expanded stem cell product can be administered following a second induction regimen or cycle thereof or a second cycle of an induction regimen, if desired or necessary. In some embodiments, a fixed dose of the expanded stem cell product can be administered following a second consolidation regimen or cycle thereof, or a second cycle of a consolidation regimen is desired or necessary. In some embodiments, a fixed dose of the expanded stem cell product can be administered following a second salvage regimen or cycle thereof, or a second cycle of a salvage regimen is desired or necessary

As discussed above, the expanded stem cell product is typically administered after the last dose of a regimen is administered, or after the last dose of each cycle of a regimen, for a regimen having more than one cycle. In some embodiments, the expanded stem cell product is administered about 12 to about 48 hours after the regimen is completed, or preferably about 24 to about 36 hours after the completion of a regimen.

In some embodiments, the expanded stem cell product is administered to the patient after the components of a chemotherapy regimen and active metabolites thereof have been cleared from the patient's blood. In some embodiments, the expanded stem cell product is administered to the patient after the components of an induction regimen and active metabolites thereof have been cleared from the patient's blood. In some embodiments, the expanded stem cell product is administered to the patient after the components of a consolidation regimen and active metabolites thereof have been cleared from the patient's blood. In some embodiments, the expanded stem cell product is administered to the patient after the components of a salvage regimen and active metabolites thereof have been cleared from the patient's blood. In some embodiments, where the chemotherapy regimen (such as an induction, salvage or consolidation regimen) is administered in more than one cycle, the expanded stem cell product is administered to the patient following each cycle after the components of the regimen and active metabolites thereof have been cleared from the patient's blood. As used herein, “after . . . regimen and active metabolites thereof have been cleared from the patient's blood” refers to clearance of the components of the regimen (e.g., a chemotherapy regimen, an induction regimen, a salvage regimen or a consolidation regimen) and active metabolites of those components that would affect the viability of CD34+ stem cells in the patient's blood, such as by decreasing CD34+ stem cell or progenitor cell viability by at least 5%, at least 10% or at least 20%.

In some embodiments, the chemotherapy regimen is an induction regimen. In some embodiments, the induction regimen is the administration of cytarabine and an anthracycline, such as daunorubicin or idarubicin. In some embodiments, the chemotherapy regimen is a “7+3” regimen of cytarabine and daunorubicin or idarubicin. The combination of cytarabine (Cytosar-U®) is given over about 4 to about 7 days and an anthracycline drug, such as daunorubicin (Cerubidine®) or idarubicin (Idamycin®), given for about 3 days is used most often. Patients may also be given hydoxyurea (Droxia®, Hydrea®) to help lower white blood cell counts.

In some embodiments, for some older adults decitabine (Dacogen™), azacitidine (Vidaza®), and low dose cytarabine may be used instead in an induction regimen.

In some embodiments, the induction regimen is GCLAC, administration of G-CSF, clofarabine and high dose cytarabine.

In some embodiments, the chemotherapy regimen is a consolidation regimen. In some embodiments, the consolidation regimen is the administration of high dose cytarabine. In some embodiments, the consolidation regimen is intermediate dose cytarabine. In some embodiments, 2-4 cycles (rounds) of high- or intermediate-dose cytarabine are administered. The expanded stem cell product can be administered after each cycle.

In some embodiments, the chemotherapy regimen is a salvage regimen. In some embodiments, the salvage regimen is the administration of cladribine, high dose cytarabine and G-CSF (CLAG). In some embodiments, the salvage regimen is a combination of etoposide, cytarabine and mitoxantrone (MEC). The expanded stem cell product can be administered after each cycle.

In other embodiments, the chemotherapy regimen can be 7+3 (7 days of Ara-C (cytarabine) plus 3 days of an anthracycline antibiotic, either daunorubicin (DA or DAC variant) or idarubicin (IA or IAC variant)); 5+2 (5 days of Ara-C(cytarabine) plus 2 days of idarubicin (IA or IAC variant); BACOD (bleomycin, doxorubicin, cyclophosphamide, vincristine, dexamethasone); CBV (cyclophosphamide, BCNU (carmustine), VP-16 (etoposide)); CHOEP (cyclophosphamide, hydroxydaunorubicin (doxorubicin), etoposide, vincristine (Oncovin®), prednisone); CEPP (cyclophosphamide, etoposide, procarbazine, prednisone); CHOP (cyclophosphamide, hydroxydaunorubicin (doxorubicin), vincristine, prednisone); CHOP—R or R-CHOP (CHOP+rituximab); CVAD and Hyper-CVAD (cyclophosphamide, vincristine, doxorubicin, dexamethasone); DA or DAC (daunorubicin×3 days plus ara-C(cytarabine)×7 days, a variant of 7+3 regimen); DAT (daunorubicin, cytarabine (ara-C), tioguanine); DHAP (dexamethasone, cytarabine (ara-C), platinum agent); DHAP-R or R-DHAP (dexamethasone, cytarabine (ara-C), platinum agent plus rituximab); DICE (dexamethasone, ifosfamide, cisplatin, etoposide (VP-16)); EPOCH (etoposide, prednisone, vincristine, cyclophosphamide, and hydroxydaunorubicin); EPOCH-R or R-EPOCH (etoposide, prednisone, vincristine, cyclophosphamide, and hydroxydaunorubicin plus rituximab); ESHAP (etoposide, methylprednisolone, cytarabine (ara-C), platinum agent); FCM or FMC (fludarabine, cyclophosphamide, mitoxantrone); FCM-R or R-FCM or R-FMC or FMC-R (fludarabine, cyclophosphamide, mitoxantrone plus rituximab); FCR (fludarabine, cyclophosphamide, rituximab); FM (fludarabine, mitoxantrone); FM-R or R-FM or RFM or FMR (fludarabine, mitoxantrone, and rituximab); FLAG (fludarabine, cytarabine, G-CSF); FLAG-Ida or FLAG-IDA or IDA-FLAG or Ida-FLAG (fludarabine, cytarabine, idarubicin, G-CSF); FLAG-Mito or FLAG-MITO or Mito-FLAG or MITO-FLAG or FLANG (mitoxantrone, fludarabine, cytarabine, G-CSF); FLAMSA (fludarabine, cytarabine, amsacrine); FLAMSA-BU or FLAMSA-Bu (fludarabine, cytarabine, amsacrine, busulfan); FLAMSA-MEL or FLAMSA-Mel (fludarabine, cytarabine, amsacrine, melphalan); GDP (gemcitabine, dexamethasone, cisplatin); GemOx or GEMOX (gemcitabine, oxaliplatin); GemOx-R or GEMOX-R or R-GemOx or R-GEMOX (gemcitabine, oxaliplatin, rituximab); GCLAC (G-CSF, clofarabine and high dose cytarabine); IA or IAC (idarubicin×3 days plus Ara-C(cytarabine)×7 days); ICE (ifosfamide, carboplatin, etoposide (VP-16)); ICE-R or R-ICE or RICE (ICE+rituximab); m-BACOD (methotrexate, bleomycin, doxorubicin (Adriamycin®), cyclophosphamide, vincristine, dexamethasone); MACOP-B (methotrexate, leucovorin (folinic acid), doxorubicin, cyclophosphamide, vincristine, prednisone, bleomycin); MINE (mesna, ifosfamide, novantrone, etoposide); MINE-R or R-MINE (mesna, ifosfamide, novantrone, etoposide plus rituximab); ProMACE-MOPP (methotrexate, doxorubicin, cyclophosphamide, etoposide and MOPP); R-Benda (rituximab+bendamustine); R-DHAP or DHAP-R (rituximab+DHAP); R-FCM or FCM-R (rituximab+FCM); R-ICE or ICE-R or RICE (rituximab+ICE); or TAD (tioguanine, cytarabine (ara-C), daunorubicin).

In some embodiments, administration of the expanded stem cell product can improve the treatment outcome of a patient having AML by improving the chance of the patient achieving a remission. In some embodiments, administration of the expanded stem cell product can improve the treatment outcome of a patient having AML by improving the chance of the patient achieving a complete response/remission (CR), e.g., a morphologic CR, cytogenetic CR, or molecular CR, or complete response/remission with incomplete blood count recovery (CRi). In some embodiments, an improved treatment outcome is other than a morphologic leukemic free state, a partial response or stable disease. In some embodiments, the improved treatment outcome is associated with an increase in IL-2 level in the patient following administration of the expanded stem cell product.

In some embodiments, the patient having AML is between about 20 and about 60 years old. In some embodiments, the patient having AML is less than 20 years old. In some embodiments, the patient having AML is greater than 60 years old or greater than 70 years old. In some embodiments, the patient having AML is greater than 60 years old or greater than 70 years old and is receiving a reduced intensity chemotherapy regimen.

The expanded stem cell product is administered as a fixed dose to a human patient in need thereof, having AML or other hematological malignancy, to improve the treatment outcome of the patient. Preferably, the expanded stem cell product is administered by infusion, such as intravenous infusion. Other suitable methods of administration of the expanded stem cell product are encompassed by the present invention. The expanded stem cell product can be administered by any convenient route, for example, by bolus injection, and can be administered together with other biologically active agents.

The fixed dose of the expanded stem cell product administered is effective in the treatment of a particular disorder or condition, such as AML or other hematological malignancy, such as for examples such as myelodysplastic syndrome (MDS), a myeloproliferative neoplasm (MPN) and non-Hodgkin Lymphoma (NHL). In some embodiments, the patient has AML, such as relapsed/refractory AML, de novo AML, or treatment-related AML. In some embodiments, the patient has a myelodysplastic syndrome (MDS), such as MDS with multilineage dysplasia (MDS-MLD); MDS with single lineage dysplasia (MDS-SLD); MDS with ring sideroblasts (MDS-RS); MDS with excess blasts (MDS-EB); MDS with isolated del(5q) or MDS, unclassifiable (MDS-U). In some embodiments, the patient has a myeloproliferative neoplasm (MPN), such as chronic myelogenous leukemia, polycythemia vera (p. vera), primary myelofibrosis, essential thrombocythemia, chronic neutrophilic leukemia, or chronic eosinophilic leukemia.

In specific embodiments, suitable fixed dosages of the expanded stem cell product for administration are about 50 million, 75 million, 100 million, 200 million, 300 million, or 400 million CD34+ cells per dose, and can be administered to a patient once, twice, three, or more times with intervals as often as needed. If the expanded stem cell product is a frozen or cryopreserved product, the number of CD34+ cells refers to the number of those cells prior to freezing or cryopreservation. In a specific embodiment, a patient receives a single fixed dose of the expanded stem cell product per regimen or per cycle of the regimen (e.g., for a multi-cycle regimen), as applicable. In a specific embodiment, a patient receives a fixed dose of the expanded stem cell product per cycle of the regimen, which administration occurs after completion of the cycle. In a specific embodiment, a patient receives a fixed dose of the expanded stem cell product per regimen, which administration occurs after completion of the regimen.

Pharmaceutical Compositions

The expanded stem cell product can be administered to a patient as a pharmaceutical (therapeutic) composition comprising a fixed dose, which is a therapeutically effective amount of expanded stem cell product, wherein administration is done without matching the HLA-types of the expanded stem cell product to the patient or to the hematopoietic stem cells or hematopoietic stem and progenitor cells in the expanded stem cell product to each other.

The present invention provides pharmaceutical compositions. Such compositions comprise a fixed dose that is a therapeutically effective amount of the expanded stem cell product, and a pharmaceutically acceptable carrier or excipient. Such a carrier can be, but is not limited to, saline, buffered saline, dextrose, water, glycerol, ethanol, and combinations thereof. The carrier and composition preferably are sterile. The formulation should suit the mode of administration. The pharmaceutical composition is acceptable for therapeutic use in humans. The composition, if desired, can also contain a pH buffering agent.

In a preferred embodiment, the composition is formulated in accordance with routine procedures as a pharmaceutical composition adapted for intravenous administration of stem cells to human beings. Typically, compositions for intravenous administration are solutions in sterile isotonic aqueous buffer. Where necessary, the composition can also include a solubilizing agent and a local anesthetic such as lidocaine to ease pain at the site of the injection.

The invention also provides a pharmaceutical pack or kit comprising one or more containers or bags filled with one or more doses of the expanded stem cell product and a diluent, such as a sterile isotonic aqueous buffer. Optionally associated with such a container(s) or bag can be a notice in the form prescribed by a governmental agency regulating the manufacture, use or sale of pharmaceuticals or biological products, which notice reflects approval by the agency of manufacture, use or sale for human administration.

In some embodiments, the present invention provides for the use of a fixed dose of an expanded stem cell product for improving treatment outcome for a patient having AML or other hematological malignancy, the expanded stem cell product comprising a pool of expanded hematopoietic stem or hematopoietic stem and progenitor cells from multiple donors, wherein the hematopoietic stem or hematopoietic stem and progenitor cells have not been HLA-matched to each other or to the patient. In some embodiments, the expanded hematopoietic stem or stem and progenitor cells are CD34+. In some embodiments, suitable fixed doses of the expanded stem cell product are about 50 million, 75 million, 100 million, 200 million, 300 million, or 400 million CD34+ cells.

### EXAMPLES

### Example 1: Generation of a Human Expanded Stem Cell Product from Human Cord Blood Units

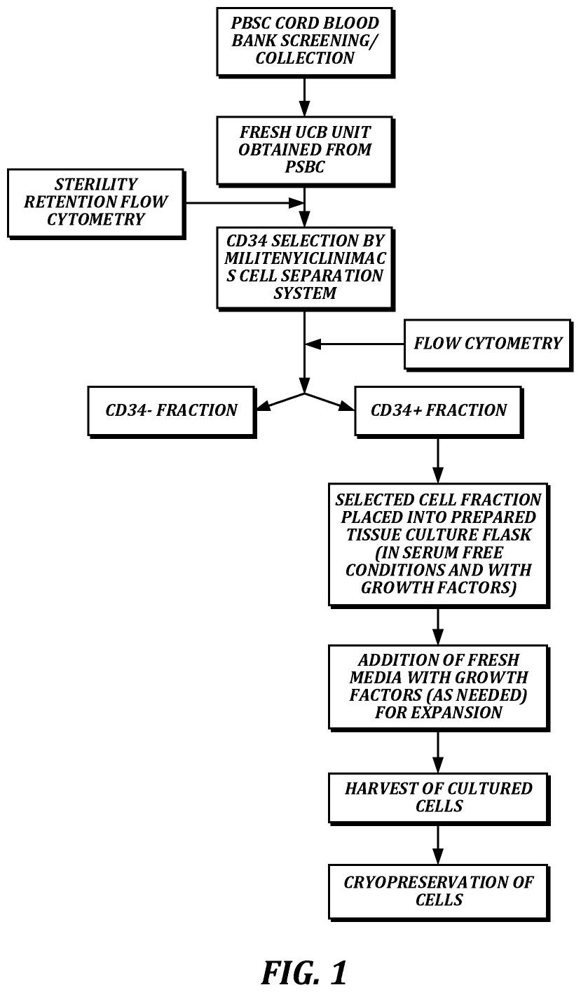

The following section describes the production and storage of an expanded stem cell product as depicted as a flow chart in FIG. 1.

Umbilical cord blood/placental blood unit(s) were collected from human donors at birth. The collected blood was then mixed with an anti-coagulant to prevent clotting. The blood was stored under quarantine at 4° C. in a monitored refrigerator. The received units were assessed, and the units to be processed for expansion was determined. The following information was collected on the units: date received, age in hours of the unit, gestational age of the donor in weeks, sex of the donor, and volume of the unit. Further, total nucleated cell count and total CD34+ cell count of each unit was determined and percent CD34+ cells was calculated. If the unit had less than 3.5 million CD34+ cells, the unit was discarded. When a unit was selected for expansion, it was removed from quarantine and assigned a unique Lot Number identifier, which it retains throughout the manufacturing process.

Prior to planned initiation of expansion cultures, tissue culture vessels were first coated overnight at 4° C. or a minimum of 2 hours at 37° C. with Delta1^{ext-IgG} at 2.5 μg/ml and RetroNectin® (a recombinant human fibronectin fragment) (Clontech Laboratories, Inc., Madison, Wis.) at 5 μg/ml in phosphate buffered saline (PBS). The flasks were then washed with PBS and then blocked with PBS-2% Human Serum Albumin (HSA). The fresh cord blood unit was processed to select for CD34+ cells using the CliniMACS® Plus Cell Separation System. Prior to CD34+ cell selection, an aliquot of the fresh cord blood unit was checked for total cell count and CD34+ cell content. Both CD34+ and CD34− cell fractions were recovered after processing. After enrichment, the percentage of CD34+ cells increased by 88- to 400-fold relative to the percentage of CD34+ cells in the sample prior to enrichment. The enriched CD34+ cell fraction was resuspended in final culture media, which consists of STEMSPAN™ Serum Free Expansion Medium II (StemCell Technologies, Vancouver, British Columbia) supplemented with rhIL-3 (10 ng/ml), rhIL-6 (50 ng/ml), rhTPO (50 ng/ml), rhFlt-3L (50 ng/ml), rhSCF (50 ng/ml).

The CD34+ enriched cells from multiple donors were added to the specifically labeled and prepared tissue culture vessels at a concentration of ≤1.8×10⁴ total nucleated cells/cm² of vessel surface area, and then placed into individually monitored and alarmed incubators dedicated solely to that lot of product. The CD34+ enriched cells were not HLA-matched to each other. After about 2 to about 4 days of culture, 50% of the original volume of fresh culture media (as above) was added to the vessels. The cell culture vessels were removed from the incubator periodically (every 1 to 3 days) and examined by inverted microscope for cell growth and signs of contamination. On about day 5 to 8, the vessel was gently agitated to mix the cells, and a 1 ml sample was removed for in process testing. The sample of cells was counted and phenotyped for expression of CD34, CD7, CD14, CD15 and CD56. Throughout the culture period, cells were transferred to additional flasks as needed when cell density increases to ≥8×10⁵ cells/ml. On the day prior to harvesting the cells for cryopreservation, fresh media was added.

On day 14, the expanded stem cell population was harvested for cryopreservation. The vessels were agitated and the entire contents transferred to sterile 500 ml centrifuge tubes. The harvested cells were centrifuged and then washed one time by centrifugation in phosphate buffered saline (PBS) and resuspended in a cryoprotectant solution containing human serum albumin (HAS), a sterile, nonpyrogenic isotonic solution of balanced electrolytes in water (Normosol-R®; Hospira, Lake Forrest, Ill.) and dimethylsulfoxide (DMSO) or CryoStor® CS10 cryopreservation medium containing 10% DMSO. Samples were taken for completion of release testing. The expanded stem cell product was frozen in a controlled-rate freezer and transferred to storage in a vapor-phase liquid nitrogen (LN2) freezer.

At the end of the culture period, the resulting cell population was heterogeneous, consisting of CD34+ stem and progenitor cells and more mature myeloid and lymphoid precursors, as evidenced by flow cytometric analysis for the presence of CD34, CD7, CD14, CD15 and CD56 antigens. There was a significant increase of CD34+ and total cell numbers during the culture period, ranging from about 100- to about 400-fold expansion of CD34+ cells and 617- to 3337-fold expansion of total cell numbers (N=9 individual cord blood units, processed per the final expansion procedures as described above). There was essentially a complete lack of T cells as measured by immunophenotyping. Functionally, these cells are capable of multi-lineage human hematopoietic engraftment in a NOD/SCID mouse model as described previously (see U.S. Patent Publication No. 2013/0095079).

### Example 2: Generation of a Human Expanded Stem Cell Product from a Frozen Human Cord Blood Unit

An expanded stem cell product containing the total cell progeny generated from enriched CD34+ cells selected from pooled human cord blood units (pool of 4 to 20 individual units) was prepared. The pooled human cord blood units were cultured in the presence of Notch ligand Delta1^{ext-IgG} (DXI) and recombinant cytokines as follows.

Cord blood units having between about 2 million to 20 million cells were selected for use. The cord blood units were thawed followed by centrifugation to remove cryoprotectant, and resuspension in a selection buffer and pooling into a single container. The selection buffer was typically PBS with 1 mM EDTA and other components. The cord blood units were typically thawed in pairs. The cells were washed twice in the selected buffer. The cells were pooled without consideration of HLA antigens or alleles (i.e., unmatched). The pooled cord blood units were pre-incubated with paramagnetic beads and then processing by CliniMACS to enrich for CD34+ cells using single use tubing sets. After selection, the cells were centrifuged, and the collected CD34+ cells were suspended in cell culture medium (StemSpan Serum Free Expansion Medium II (SFEMII) media supplemented with 5 recombinant human cytokines IL-3 (10 ng/ml), and IL-6, TPO, SCF, and Flt-3L (each at 50 ng/ml)). The enriched CD34+ cells were then sampled to determine viable cell yield and percentage of CD34+ cells in the composition. CD34+ cells were placed into coated flasks at a suitable target seed density using StemSpan SFEMII media supplemented with 5 recombinant human cytokines (IL-3, IL-6, TPO, SCF, Flt-3L)). Prior to use, the flasks were coated with the recombinant proteins DXI (2.5 micrograms/me and RetroNectin® recombinant human fibronectin fragment (rFN-CH-206) (5 micrograms/me; unbound protein was washed from the flasks prior to use. The flasks were fed fresh SFEMII media and cytokines, as needed. When the cells reached a sufficient cell number, the cells were harvested, pooled, and passaged into larger vessels at a suitable target seed density using the same SFEMII media and 5 cytokines. The vessels were also pre-coated overnight with the DXI and RetroNectin® recombinant human fibronectin fragment (rFN-CH-206), as described above. The vessels were monitored for cell density and viability and fed up to a full volume of fresh SFEMII media and cytokines, as needed. When the cells reach the desired cell density, the cells were harvested by slight agitation, concentrated by centrifugation, the media is removed, and the cells resuspended into wash buffer. The viable CD34+ cell count was determined. After washing and harvesting, the cell pellets were resuspended in a balanced electrolyte solution with albumin. The final stem cell product typically contained about 50 to about 100 million cells/ml.

The final cell product was then added to cryoprotectant media, followed by aseptic filling into labeled CryoStore bags, cryopreservation in a controlled rate freezer, and storage in a vapor-phase liquid nitrogen (LN2) freezer at <−150° C. The bags are filled with from about 50 to about 400 million CD34+ cells in a volume of about 20 ml/bag. The cryoprotectant media contained about 4% human serum albumin (HSA), 10% dimethylsulfoxide (DMSO), in Normosol-R or CyroStor® CS10 as described.

### Example 3: Treatment of Patients Having AML with an Expanded Stem Cell Product

Patients with acute myeloid leukemia undergoing intense myelosuppressive chemotherapy regimens are at risk for life-threatening infections that impact overall treatment outcomes. The use of a non-HLA matched, pooled cord-blood-derived ex vivo expanded CD34+ stem cell product (dilanubicel or NLA101) on the rate of severe bacterial or fungal infections was investigated in phase I and phase 2 studies. Dilanubicel was administered in conjunction with induction and consolidation chemotherapy. A global phase 2 randomized open-label study enrolled 146 of a planned 220 subjects into one of 4 treatment arms: standard of care (SOC) alone or SOC plus low, medium, or high dose dilanubicel (100×10⁶, 300×10⁶, or 800×10⁶ CD34+ cells, respectively). Up to 3 doses of dilanubicel could be given with each round of chemotherapy, and subjects were followed for up to 84 days or 30 days after last dose of chemotherapy or dilanubicel. When the study was halted no particular effect was seen on infection rates, surprisingly, dilanubicel-treated subjects experienced higher complete response (CR) rates as compared to patients on the control arm who received chemotherapy alone. In addition, treatment with dilanubicel was associated with a transient dose-dependent increase in serum Interleukin-2 (IL-2) levels. There were no Data Safety Monitoring Board-related safety concerns, but a few unexpected serious adverse events (SAEs) were observed. However, neither graft-versus-host disease or cytokine release syndrome was not observed.

Materials and Methods

Trial design: This study was a phase 2 open-label, multi-center, randomized, controlled, dose-finding study of the safety and efficacy of dilanubicel to reduce the rate of infections associated with chemotherapy-induced neutropenia in adult subjects with AML. This study was conducted in 36 sites in the United States (US), South Korea (SK), and Australia (AU). Following enrollment, subjects were randomized 1:1:1:1 to either the control arm (Standard of Care (SOC) chemotherapy) or 1 of 3 investigational arms (SOC chemotherapy+low dose, medium dose, or high dose dilanubicel). Randomization was stratified by geographic region (US vs SK/AU).

Subjects randomized to an investigational arm were eligible to receive a single fixed assigned dose of dilanubicel after the first cycle of chemotherapy, and up to 2 additional doses after subsequent chemotherapy cycles (one infusion per cycle). Subjects randomized to the SOC arm were treated comparably, but without infusion of dilanubicel for up to 3 cycles of chemotherapy. All subjects were to be followed for 84 days following randomization, or 30 days post final infusion of dilanubicel, or 30 days post the day after the last chemotherapy infusion for SOC Arm, whichever was longer. The study was halted after enrollment of 146 subjects (66%) after completion of an unplanned interim analysis.

The protocol and its amendments were approved by the relevant institutional review boards and ethics committees and required written informed consent prior to any study procedures. Safety was overseen by an independent Data Safety Monitoring Board (DSMB).

Patients: Eligible subjects must have had untreated de novo or secondary AML and planned to receive at least 2 cycles of chemotherapy with curative intent per local institutional standards. Induction chemotherapy was required to contain an anthracycline and cytarabine backbone and be expected to lead to moderate to severe myelosuppression. Subjects were also required to have a Karnofsky score ≥50 or Eastern Cooperative Oncology Group (ECOG) performance status of 0, 1, or 2, and were required to have adequate renal, hepatic, pulmonary, and cardiac function and be without evidence of active uncontrolled infection at screening. Concomitant use of granulocyte transfusions, immunotherapy, other investigational agents was exclusionary.

Study treatment: Dilanubicel is an ex vivo expanded hematopoietic stem and progenitor cell (HSPC) product derived from pooled, unmatched cord blood derived CD34+ cells as described above. CD34+ cells were isolated from qualified screened cord blood donors and cultured for 16 days in the presence of immobilized Notch ligand and recombinant cytokines (generally as described above). The expanded stem cell product was cryopreserved until infusion and provided to the study sites in fixed doses of approximately 100 million (low dose), 300 million (medium dose), and 800 million (high dose) CD34+ cells/bag in a volume of approximately 20 ml. Dilanubicel was given intravenously over 5 to 10 minutes approximately 24 to 36 hours after last dose of chemotherapy for a given cycle. Dosing was immediately preceded by administration of an oral acetaminophen and an intravenous antihistamine.

Endpoints and statistical analyses: The primary endpoint of the study was rate of severe (Common Terminology Criteria for Adverse Events (CTCAE) grade 3 or higher) bacterial or fungal infections over the course of the 84-day study period. The analysis was performed by counting the number of unique Grade ≥3 infections within a subject and normalizing by the number of days on study. The normalized infection rate was regressed on treatment arm (SOC as reference) and geographical region using a negative binomial regression in order to compare the infection rates between treatment arms. Number of days on study from study day 1 was used as an offset variable to account for differential follow-up due to death or loss to follow-up. Event rate ratios and 95% confidence intervals were calculated as a measure of strength of association and precision respectively.

Key secondary endpoints included best overall treatment response, use of filgrastim, and incidence and duration of febrile neutropenia, and safety. Treatment response was defined as a complete remission (CR) or complete remission with incomplete count recovery (CRi) per Revised International Working Group criteria, and treatment arms were compared using a Cochran-Mantel-Hansel test, stratified by geographical region.

Results

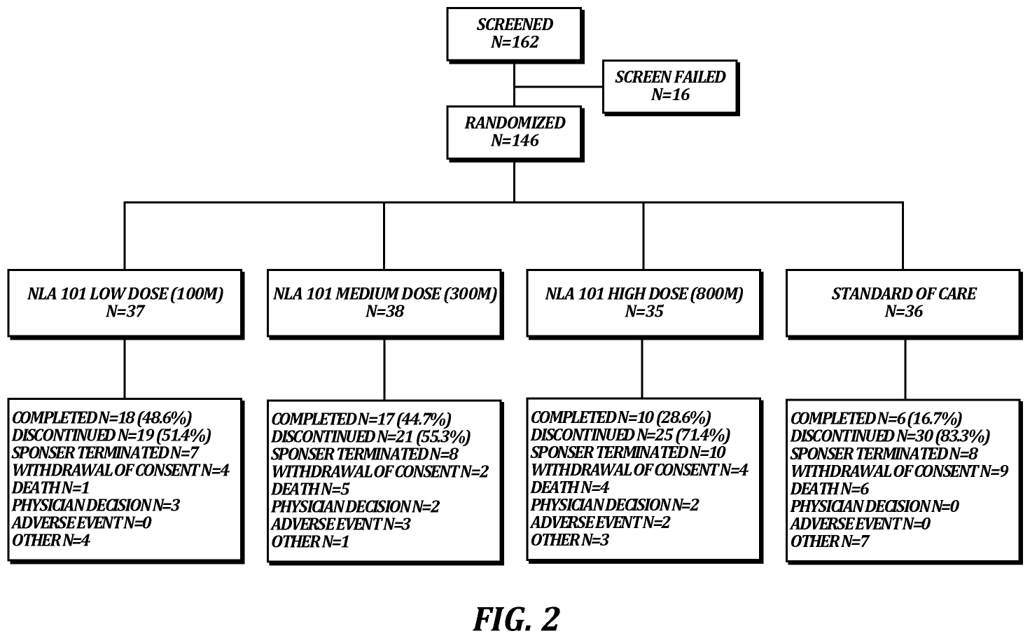

Of the 162 subjects screened for this study prior to enrollment closure, 146 were enrolled and randomized to receive study treatment: 37 to the Low Dose arm, 38 to the Medium Dose arm, 35 to the High Dose arm, and 36 were randomized to the SOC arm. At the time the study halted, 18 subjects (48.6%) in the Low Dose arm, 17 subjects (44.7%) in the Medium Dose arm, 10 subjects (28.6%) in the High Dose arm, versus 6 (16.7%) subjects in the SOC arm had completed the study per protocol. A summary of subject disposition is provided in FIG. 2. The number of subjects treated with dilanubicel was 33 (89.2%) in the Low Dose arm, 34 (89.5%) in the Medium Dose arm, and 34 (97.1%) in the High Dose arm. The median number of dilanubicel doses received per subject was 2 in all 3 arms.

The overall median age of randomized subjects was 60 years (range, 19-77). The sex of subjects was evenly split, male (75 subjects; 51.4%) versus female (71 subjects, 48.6%). The majority of subjects were white (110 subjects; 75.3%). Most baseline disease characteristics were relatively balanced among groups although a higher percentage of subjects had unfavorable risk AML in the SOC arm.

The total number of Grade ≥3 bacterial or fungal infections that occurred during the study period was 23 in the Low Dose arm, 22 in the Medium Dose arm, 25 in the High Dose arm, and 20 in the SOC arm. The test of total dilanubicel versus SOC was not statistically significant, p=0.9604. The rate ratios and associated 95% CIs for each treatment arm versus SOC arm were 0.88 (0.44, 1.77; p=0.7291) Low Dose, 0.93 (0.46, 1.88; p=0.8471) Medium Dose, and 1.05 (0.53, 2.09; p=0.8868) High Dose.

Table 1 summarizes the best overall response rate of complete remission (CR) (including morphologic CR, cytogenetic CR, molecular Cr or CRi) versus Not CR (all other non-CR response assessments) by treatment arm over the course of the study. Each treatment arm had numerically favorable CR rates compared to the SOC arm, which was statistically significant in the Medium Dose arm (p=0.0024) and the total treatment group (p=0.0086). This observation was unexpected, particularly in view of a prior study (Delaney et al., 2016, Lancet 3(7):PE330-339) using a single donor, unmatched expanded cord blood product. Although CR rate was not a primary endpoint of the prior study, an increase in CR rate was not reported.

TABLE 1
Complete Response/Remission Rate By Treatment Group

| | Low Dose | Medium | | High Dose | Total | |
|---|---|---|---|---|---|---|
| | (100M) | Dose (300M) | | (800M) | NLA101 | SOC |
| | (N = 37) | (N = 38) | | (N = 35) | (N = 110) | (N = 36) |
| | n (%) | n (%) | | n (%) | n (%) | n (%) |
| Number with Assessment | (n = 30) | (n = 32) | | (n = 35) | (n = 97) | (n = 30) |
| CR | 18 (60.0%) | 25 | (78.1%) | 22 (62.9%) | 65 (67.0%) | 12 (40.0%) |
| Not CR | 12 (40.0%) | 7 | (21.9%) | 13 (37.1%) | 32 (33.0%) | 18 (60.0%) |

| | | | | | |
|---|---|---|---|---|---|
| p-value vs SOC* | 0.1247 | 0.0024 | 0.0703 | 0.0086 | N/A |

Note:
CR = Morphologic CR, Cytogenetic CR, Molecular CR, or CR with incomplete blood count recovery (CRi);
Not CR = Morphologic leukemia-free state, Partial remission/response, Early Assessment, Treatment Failure
*p-values from a CMH test stratified by geographical region.

Overall dilanubicel was generally well tolerated with a dose dependent increase in related events, although overall incidence of safety events in the High Dose arm was only modestly higher than in the SOC arm. The most common adverse events assessed as related to dilanubicel were fever/febrile neutropenia, infusion reactions, and inflammatory signs and symptoms. The occurrence of death in the study was not elevated in any expanded stem cell product arm compared to the SOC arm. The DSMB monitored safety throughout the study and raised no safety concerns. The numerically favorable CR rates in each treatment arm compared to the SOC arm was unexpected.

### Example 4: Treatment of Patients Having a Hematological Malignancy with an Expanded Stem Cell Product

Patients having a hematological malignancy and undergoing intense chemotherapy regimen are treated with standard of care (SOC) plus low, medium, or high dose dilanubicel (100×10⁶, 300×10⁶, or 800×10⁶ CD34+ cells, respectively). Dilanubicel is administered after each cycle of the chemotherapy. The patients are followed per standard practice and the best overall response rate of complete remission (CR) (including morphologic CR, cytogenetic CR, molecular Cr or CRi) versus Not CR (all other non-CR response assessments) is assessed over the course of the study.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications, including patents, patent application publications, and scientific literature, are cited herein, the disclosures of which are incorporated by reference in their entireties for all purposes.

The embodiments of the invention in which an exclusive property or privilege is claimed are defined as follows:

1. A method of improving treatment outcome for a human patient having acute myelogenous leukemia (AML), comprising:
administering an induction chemotherapy regimen to the patient;
administering a fixed dose of a CD34+ enriched, T cell depleted, expanded stem cell product to the patient after the administration of the induction regimen; wherein the expanded stem cell product comprises hematopoietic stem cells or hematopoietic stem and progenitor cells derived from cord blood units from at least two different human donors, wherein the cord blood units are selected without matching to the HLA type of the donors and without matching to the HLA type of the human patient;
monitoring the status of the patient to determine whether the patient has achieved a remission; and
administering a second induction chemotherapy regimen followed by administration of a second fixed dose of the expanded stem cell product to the patient, if the patient has not achieved a remission.

2. The method of claim 1, further comprising administering a consolidation chemotherapy regimen to the patient that has achieved a remission, followed by administration of a fixed dose of the expanded stem cell product.

3. The method of any one of claim 1 or claim 2, wherein each dose of the expanded stem cell product is administered about 12 to 48 hours, or preferably about 24 to 36 hours, after the induction chemotherapy regimen.

4. The method of claim 1, wherein each dose of the expanded stem cell product is administered to the patient after the components of the induction chemotherapy regimen and active metabolites thereof have cleared from the patient's blood.

5. The method of claim 2, wherein the dose of the expanded stem cell product is administered about 12 to 48 hours, or preferably about 24 to 36 hours, after the consolidation chemotherapy regimen

6. The method of claim 2, wherein the dose of the expanded stem cell product is administered after the components of the consolidation chemotherapy regimen and active metabolites thereof have been cleared from the patient's blood.

7. The method of claim 1, wherein the induction chemotherapy regimen comprises administration of a combination of cytarabine and an anthracycline.

8. The method of claim 7, wherein the anthracycline is daunorubicin or idarubicin.

9. The method of claim 7, wherein the induction regimen is a 7+3 regimen.

10. The method of claim 7, wherein the induction chemotherapy regimen comprises administration of cytarabine and daunorubicin.

11. The method of claim 2, wherein the consolidation chemotherapy regimen comprises administration of an intermediate dose or a high dose cytarabine.

12. The method of claim 1, further comprising administering a salvage chemotherapy regimen.

13. The method of claim 12, wherein the salvage chemotherapy regimen comprises cladribine, high dose cytarabine and G-CSF (CLAG) or etoposide, cytarabine and mitoxantrone (MEC).

14. The method of any of the preceding claims, wherein each fixed dose of the expanded stem cell product comprises from about 50 million CD34+ cells to about 400 million CD34+ cells.

15. The method of any of the preceding claims, wherein each fixed dose of the expanded stem cell product comprises from about 100 million to about 300 million CD34+ cells.

16. The method of any of any of the preceding claims, wherein:
a. each fixed dose of the expanded stem cell product is the same;
b. each fixed dose of the expanded stem cell product administered following the induction chemotherapy regimen is the same; or
c. each fixed dose of the expanded stem cell product administered following the induction chemotherapy regimen is different than the fixed dose administered following the consolidation chemotherapy regimen.

17. The method of any of the preceding claims, wherein the expanded stem cell product further comprises a cryoprotective agent.

18. The method of any of the preceding claims, wherein the expanded stem cell product was produced by steps comprising enriching for CD34+ human cord blood stem and progenitor cells and expanding the CD34+ enriched human cord blood stem and progenitor cells with a Notch agonist.

19. The method of claim 18, wherein the Notch agonist is an extracellular domain of the Delta fused to the Fc portion of IgG (Delta^{ext-IgG}).

20. The method of any of the preceding claims, wherein the expanded stem cell product is derived from cord blood units from at least four different human donors, from at least six different human donors, or from at least eight different human donors.

21. The method of any of the preceding claims, wherein the expanded stem cell product does not transiently engraft in the patient at day 14 after administration.

22. The method of any of the preceding claims, wherein the monitoring step comprises determining whether patient has <5% marrow blasts by morphology.

23. The method of any of the preceding claims, wherein following the induction regimen and prior to the consolidation regimen, the patient does not receive a cord blood unit that is at least partially matched to the HLA-type patient.

24. The method of claim 23, wherein the at least partially matched cord blood unit is an autologous transplant, a haploidentical transplant, a matched related donor transplant, a matched unrelated donor transplant, or a mismatched unrelated donor transplant.

25. A method of improving treatment outcome for a human patient having a hematological malignancy, comprising:
administering a chemotherapy regimen to the patient;
administering a fixed dose of an expanded stem cell product to the patient after the administration of the chemotherapy regimen; wherein the expanded stem cell product comprises hematopoietic stem cells or hematopoietic stem and progenitor cells derived from at least two different human donors, wherein the hematopoietic stem cells or hematopoietic stem and progenitor cells are selected without matching to the HLA type of the donors and without matching to the HLA type of the human patient;
monitoring the status of the patient to determine whether the patient has achieved a remission; and
optionally administering a second chemotherapy regimen followed by administration of another fixed dose of the expanded stem cell product to the patient, if the patient has not achieved a remission.

26. The method of claim 25, wherein each dose of the expanded stem cell product is administered about 12 to 48 hours, or preferably about 24 to 36 hours, after the chemotherapy regimen.

27. The method of claim 25, wherein each dose of the expanded stem cell product is administered to the patient after the components of the chemotherapy regimen and active metabolites thereof have been cleared from the patient's blood.

28. The method of any one of claims 25-28, wherein the hematological malignancy is selected from acute myelogenous leukemia (AML), a myelodysplastic syndrome (MDS), Non-Hodgkin lymphoma (NHL) and a myeloproliferative neoplasm (MPN).

29. The method of claim 28, wherein the AML is de novo acute myelogenous leukemia (AML), relapsed/refractory AML or treatment-related AML.

30. The method of claim 28, wherein the MDS is selected from MDS with multilineage dysplasia (MDS-MLD); MDS with single lineage dysplasia (MDS-SLD); MDS with ring sideroblasts (MDS-RS); MDS with excess blasts (MDS-EB); MDS with isolated del(5q); and MDS, unclassifiable (MDS-U).

31. The method of claim 28, wherein the MPN is selected from chronic myelogenous leukemia, polycythemia vera (p. vera), primary myelofibrosis, essential thrombocythemia, chronic neutrophilic leukemia, or chronic eosinophilic leukemia.

32. The method of any one of claims 25 to 31, wherein the expanded stem cell product comprises hematopoietic stem cells or hematopoietic stem and progenitor cells derived from cord blood units from at least two different human donors.

33. The method of any one of claims 25 to 32, wherein the chemotherapy regimen is selected from an induction regimen, a salvage regimen, and a consolidation regimen.

34. The method of any one of claims 25 to 33, wherein the chemotherapy regimen is an induction regimen comprising administration of cytarabine and an anthracycline.

35. The method of claim 34, wherein the anthracycline is daunorubicin or idarubicin.

36. The method of claim 34, wherein the induction regimen is a 7+3 regimen.

37. The method of any of claims 25 to 33, wherein the chemotherapy regimen is a consolidation regimen comprising administration of intermediate dose or high dose cytarabine.

38. The method of claim 37, wherein the chemotherapy regimen is a salvage regimen.

39. The method of any one of claims 25 to 33, wherein the salvage regimen is cladribine, high dose cytarabine and G-CSF (CLAG) or etoposide, cytarabine and mitoxantrone (MEC).

40. The method of any of one of claims 25 to 39, wherein each fixed dose of the expanded stem cell product comprises from about 50 million CD34+ cells to about 400 million CD34+ cells.

41. The method of any one of claims 25 to 40, wherein each fixed dose of the expanded stem cell product comprises from about 100 million to about 300 million CD34+ cells.

42. The method of any one of claims 25 to 41, wherein each fixed dose of the expanded stem cell product is the same.

43. The method of any of claims 25 to 42, wherein the expanded stem cell product further comprises a cryoprotective agent.

44. The method of any one of claims 25 to 43, wherein the expanded stem cell product was produced by steps comprising enriching for CD34+ human cord blood stem and progenitor cells and expanding the CD34+ enriched human cord blood stem and progenitor cells with a Notch agonist.

45. The method of claim 44, wherein the Notch agonist is an extracellular domain of the Delta fused to the Fc portion of IgG (Delta^{ext-IgG}).

46. The method of any of the preceding claims, wherein the expanded stem cell product is derived from cord blood units from at least four different human donors, from at least six different human donors, or from at least eight different human donors.

47. The method of any one of claims 25 to 46, wherein the expanded stem cell product does not transiently engraft in the patient, as determined at day 14 after administration.
